(19) United States
(12) Patent Application Publication
(10) Pub. No.: US 2015/0143913 A1
(43) Pub. Date: May 28, 2015

(54) MULTI-MODAL SENSING FOR VEHICLE

(71) Applicant: Purdue Research Foundation, (US)

(72) Inventors: Douglas Edward Adams, West Lafayette, IN (US); John Stuart Bolton, West Lafayette, IN (US); Edward J. Delp, III, West Lafayette, IN (US)

(73) Assignee: Purdue Research Foundation, West Lafayette, IN (US)

(21) Appl. No.: 13/747,005

(22) Filed: Jan. 22, 2013

Related U.S. Application Data
(60) Provisional application No. 61/588,233, filed on Jan. 19, 2012.

(51) Int. Cl. G01N 33/22; G01H 1/00; G06K 9/00; G01H 9/00

(52) U.S. Cl. CPC G01N 33/227; G01H 9/00; G01H 1/00; G06K 9/00771
USPC 73/655; 73/649; 382/104

(57) ABSTRACT

Apparatuses and methods for detecting hidden or modified objects in a vehicle are disclosed. Embodiments include detection of vehicle irregularities, such as irregularities in the acoustical response of a vehicle tire as the tire rolls over a surface, irregularities in the orientation of the vehicle body, the response of a vehicle component (such as an axle, wheel, tire, etc.) to the vehicle moving over a surface, and the response of a vehicle component to vehicle generated vibrations. In alternate embodiments subsystems detecting different irregularities communicate with one another.

This application claims the benefit of U.S. Provisional Application No. 61/588,233, filed Jan. 19, 2012, the entirety of which is hereby incorporated herein by reference.

### GOVERNMENT RIGHTS

This invention was made with U.S. Government support under Grant No. N00173-09-1-G901 by Naval Research Laboratory. The U.S. Government has certain rights in the invention.

### SUMMARY

Embodiments of the present disclosure provide methods and apparatuses for sensing, detecting, and/or analyzing vehicles and the characterization of a vehicle.

Embodiments of the present disclosure provide methods and apparatuses for detecting irregularities and/or anomalies of a vehicle, certain embodiments detecting the presence of hidden materials, such as drugs, hidden persons, explosives, and general contraband. The feasibility of several technologies for detecting vehicle-borne improvised explosive devices (VBIEDs) in the form of anomalous payloads have been evaluated. Theoretical simulations and experimental measurements were conducted to gauge the effectiveness of each technology in assessing unusual payloads in vehicles passing through a checkpoint. Simulations were used to discover patterns in the response that are particularly sensitive to anomalous payloads in a moving vehicle undergoing dynamic response. Experiments were used to ascertain the sensitivity of certain measurements to these payloads.

Based on the results obtained, it has been determined that 1) video analytics performed on the car and driver, 2) static and dynamical analysis of the vehicle wheels and chassis, and 3) acoustical analysis of the tire could provide, when combined into a single check point, the means of detecting anomalous payloads stowed in vehicle cavities. The fusion of these detection methods can:
Indicate vehicle make, model, and dimensions for use in comparing other vehicle measurements to those available in a large database of vehicles;
Indicate vehicle mass and loading for use in comparing to a database of vehicles;
Indicate tire size, interior pressure, and interior medium to detect over inflated or filled tires;
Indicate abnormal driver behavior as it correlates to the intent to deceive the checkpoint.

Each technology was explored by starting with an investigation of previous research for the detection of VBIEDs or vehicle payloads using conventional methods such as weigh stations. Viable options were further analyzed and experimental measurements were acquired to verify that the methods were feasible. For example, the increase in the percentage of anomalous vehicle payloads detected based on a Canadian vehicle database was estimated given additional levels of information regarding the vehicle that could be obtained through video analytics. This analysis provides an indication of the more fruitful investment from a standpoint of vehicle payload detectability.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein is not necessarily intended to address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

Some of the figures shown herein may include dimensions or may have been created from scaled drawings. However, such dimensions, or the relative scaling within a figure, are by way of example, and not to be construed as limiting.

FIG. 3.2: Overall Process Flow according to one embodiment of present disclosure.

FIG. 3.3.1: Block diagram of the video analysis unit system according to one embodiment of present disclosure.

FIG. 3.3.2: Video detection tasks and their interdependence according to one embodiment of present disclosure.

FIG. 3.3.3: Overview of camera deployment according to one embodiment of present disclosure.

FIG. 3.3.4: Vehicle detection by background subtraction according to one embodiment of present disclosure.

FIG. 3.3.6: Representative vehicles and their silhouettes for four classes according to one embodiment of present disclosure.

FIG. 3.3.7: Vehicle body type determination according to one embodiment of present disclosure.

FIG. 3.3.8: A machine's view of a moving vehicle's tire with shiny hub according to one embodiment of present disclosure.

FIG. 3.3.9: A model of the wheel-rubber edge used for size estimation according to one embodiment of present disclosure.

FIG. 3.3.10: Final step in tire size estimation according to one embodiment of present disclosure.

FIG. 3.3.11: A machine's view of a vehicle's tire with dark hub according to one embodiment of present disclosure.

FIG. 3.3.12: Extraction of vehicle's tire for size estimation according to one embodiment of present disclosure.

FIG. 3.3.13: Examples of successful Make recognition according to one embodiment of present disclosure.

FIG. 3.3.14: Vehicle tracking in the absence of traffic according to one embodiment of present disclosure.

FIG. 3.3.15: Vehicle tracking the presence of traffic according to one embodiment of present disclosure.

FIG. 3.3.16: Vehicle trajectories obtained under different driving types according to one embodiment of present disclosure.

FIG. 3.3.17: Vehicle trajectories transformed to ground distances according to one embodiment of present disclosure.

FIG. 3.3.18: Analyzed trajectories under three types of driving scenarios according to one embodiment of present disclosure.

FIG. 3.4.1 5 DoF ½ car model according to one embodiment of present disclosure.

FIG. 3.4.4: Transient response to added weight according to one embodiment of present disclosure.

FIG. 3.4.5: Force in Tires in Response to Cleat Excitation with Load Stiffness=10,000 N/m according to one embodiment of present disclosure.

FIG. 3.4.6: Force output in tires in response to cleat excitation with kL=100,000 N/m according to one embodiment of present disclosure.

FIG. 3.4.8: First natural frequency as a function of kr, kt, a, and lcm according to one embodiment of present disclosure.

FIG. 3.4.09: Illustration of apparatus and system for measuring ground vehicle dynamic response through wheels according to one embodiment of the present disclosure.

FIG. 3.4.11: Feature vectors for loaded and unloaded vehicles according to one embodiment of present disclosure.

FIG. 3.4.11_A: Filtered angular acceleration of loaded and unloaded signals according to one embodiment of present disclosure.

FIG. 3.4.12: Sensitivity analysis results for excess vehicle detection parameters according to one embodiment of present disclosure.

FIG. 3.5.3: First modal frequency variation with respect to the percent mass change according to one embodiment of present disclosure.

FIG. 3.5.5: (a) Unloaded Door Panel (b) Loaded with 8 lbs sand bag (c) Loaded with 16 lbs sand bag (d) Loaded with 24 lbs sand bags (e) Loaded with 32 lbs sand bags according to one embodiment of present disclosure.

FIG. 3.5.7: analysis mode shape and experimental mode shape according to one embodiment of present disclosure.

FIG. 3.5.9: Amplitude integration technique comparison for the 5 different cases over 3 different channels according to one embodiment of present disclosure.

FIG. 3.5.10: Integration techniques for each point and loading condition. “# of points on door” represents the number of the 45 points which were included in the integration. Points 0-15 lay on the first row of door grid points, 16-30 on the second row, and 31-45 on the third row. The mass increase indicates 5 loading conditions discussed previously according to one embodiment of present disclosure.

FIG. 3.5.11: Mode shift of loaded car door. Channel 1 is located at the top of the door; channel 3 is located at the bottom of the door according to one embodiment of present disclosure.

FIG. 3.6.1: Mode splitting/orientation due to tire loading according to one embodiment of present disclosure.

FIG. 3.6.2: Insulation filling inserted into tire to eliminate tire acoustical mode according to one embodiment of present disclosure.

FIG. 3.6.3: Comparison between position-frequency (left) and wavenumber-frequency (right). The lines in the wavenumber-frequency plot indicate propagating waves according to one embodiment of present disclosure.

FIG. 3.6.4: Frequency-wavenumber decomposition for tire sidewall at 30 psi according to one embodiment of present disclosure.

FIG. 3.6.5: Frequency-wavenumber decomposition for tire sidewall at 55 psi according to one embodiment of present disclosure.

FIG. 3.6.6: Frequency-wavenumber decomposition detail for tire sidewall at 30 psi. Features related to the tire acoustical mode are highlighted according to one embodiment of present disclosure.

FIG. 3.6.7: Frequency-wavenumber decomposition detail for tire sidewall at 55 psi. Features related to the tire acoustical mode are highlighted according to one embodiment of present disclosure.

FIG. 3.6.8: Acoustical radiation measurement setup according to one embodiment of present disclosure.

FIG. 3.6.9: Tire axle vibration signature comparisons at 60 psi according to one embodiment of present disclosure.

FIG. 3.6.10: Tire axle vibration signature comparisons at 40 psi according to one embodiment of present disclosure.

FIG. 3.6.11: Tire axle vibration signature comparisons at 20 psi according to one embodiment of present disclosure.

FIG. 3.6.12: Average acoustical radiation comparison for 60 psi tire inflation according to one embodiment of present disclosure.

FIG. 3.6.13: Average acoustical radiation comparison for 40 psi tire inflation according to one embodiment of present disclosure.

FIG. 3.6.14: Acoustical radiation with position for the 40 psi, air-filled tire according to one embodiment of present disclosure.

FIG. 3.6.15: Acoustical radiation with position for the 40 psi, insulation-filled tire according to one embodiment of present disclosure.

FIG. 3.6.16: Drop test acoustical signatures for both tires at 60 psi according to one embodiment of present disclosure.

FIG. 3.6.17: Drop test acoustical signatures for both tires at 40 psi according to one embodiment of present disclosure.

FIG. 3.6.18: Drop test acoustical signatures for both tires at 20 psi according to one embodiment of present disclosure.

FIG. 3.7.1: Block diagram of a computing system adapted for multi-modal sensing of a vehicle.

FIG. 3.7.2: Schematic diagram of a computer used in various embodiments.

### DETAILED DESCRIPTION OF THE ILLUSTRATED EMBODIMENTS

For the purposes of promoting an understanding of the principles of the disclosure, reference will now be made to one or more embodiments illustrated in the drawings and specific language will be used to describe the same. It will nevertheless be understood that no limitation of the scope of the disclosure is thereby intended; any alterations and further modifications of the described or illustrated embodiments, and any further applications of the principles of the disclosure as illustrated herein are contemplated as would normally occur to one skilled in the art to which the disclosure relates. At least one embodiment of the disclosure is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments, other embodiments may not include those same advantages, or may include different advantages. Any advantages described herein are not to be construed as limiting to any of the claims.

Specific quantities (spatial dimensions, temperatures, pressures, times, force, resistance, current, voltage, concentrations, wavelengths, frequencies, heat transfer coefficients, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

The Department of Homeland Security (DHS) must ensure their research and development efforts are going to meet the myriad needs of the many agencies within DHS. For example, Customs and Border Control requires technology that can rapidly inspect the thousands of vehicles that enter the United States for drugs, contraband, hidden persons, etc.; whereas, the Transportation Security Administration must ensure the vehicles entering airports are not laden with improvised explosives. The premise of this work is that the types of anomalies that comprise vehicle borne improved explosives (VBIEDs) will not be amenable for detection using a single sensor technology. For example, it is difficult to identify explosives within a vehicle using chemical spectrometry methods due to the containment of explosives in the cavities of the vehicle. Vehicle x-rays might be useful for detecting large payloads within a vehicle, but traffic must be slowed down to 10 miles per hour to make these measurements and officers must inspect the scans. In addition, the negative public perception of x-rays is a concern for mass deployment. However, multiple sensor technologies can be used together to detect anomalies (in payload or driver vehicle) in moving vehicles. Embodiments of the present disclosure include apparatuses and methods for detecting vehicle borne improvised explosive devices (VBIED) in the form of anomalous payloads using video sensing, vehicle dynamic sensing, body panel sensing, and acoustic sensing technologies.

In a first phase, modeling and experimentation of the vehicle dynamics, door panel vibration, video sensing, and acoustic sensing has been used to explore several potential methods for detecting anomalies. This portion of the application contains the following information:
Tasks performed and results of these tasks;
A VBIED (anomaly) detection process-flow diagram;
Video sensing methods and results;
Vehicle dynamic modeling and sensing results;
Door panel modeling and sensing results; and
Tire acoustic modeling and sensing results.

A process-flow diagram is presented that illustrates how each of these technologies work together in certain embodiments, and the remaining sections review sensing technologies that have been examined. One issue examined in this first phase of research is the sensitivity associated with each of these technologies and how the other sensing technologies can compensate for weaknesses in any one of these particular sensing technologies.

Listed in Table 3.1 are example tasks that may be included in embodiments of the present disclosure.

### 3.1 Tasks Performed and Key Results

Tasks Performed: Video sensing

| Tasks | Results/Notes |
|---|---|
| Literature survey | Surveillance is used on highways, at intersections |
| on surveillance | and in parking areas |
| of vehicles | Methods include magnetic loops, radar speed guns |
| | and video cameras |
| | Most systems require human supervision |
| Selection of the | Two video cameras-front view (FVC) and side view |
| video system's | (SVC) |
| parameters | Information from the two cameras is jointly |
| | processed-complementary roles |
| | Positions of the cameras selected based on |
| | experimental results |
| Robust vehicle | Many techniques available for object detection in |
| detection | video sequences-background subtraction, optical |
| | flow and model-based methods |
| | Motion-assisted background subtraction technique |
| | Robust to small perturbations in background and |
| | gradual illumination changes |
| | Model update is typically rare, hence fast operation |
| Vehicle | Features include aspect ratio, shape, edge orientation |
| body-type | and dimensions |
| determination | Select one of four classes (sedan, truck, SUV and |
| | hatchback) based on shape or silhouette |
| | Directly uses the output of vehicle detection |
| Tire size and | Select the circular edge between the rubber and the |
| wheelbase | wheel as an invariant feature |
| estimation | Search for the edge over space and size |
| | For tires with dark hub, additional information is |
| | needed-horizontal location of the tire center |
| Determination | Object tracking methods include repeated object |
| of trajectory | detection, mean-shift tracker, Kalman filter and |
| by tracking | particle filter |
| | Tracking using background subtraction |
| | Feature based tracking using particle filter |
| Trajectory | Map the image position to ground co-ordinates |
| analysis | Methods include geometric transforms, camera |
| for anomaly | calibration and use of fiducial markers |
| detection | Can calibrate camera at a constant speed and |
| | generate a look-up table based transform |
| | Abrupt changes in the trajectory can signal anomaly |

Tasks Performed: Vehicle Dynamics sensing

| Tasks | Results/Notes |
|---|---|
| ½ car Matlab model | Increasing payloads cause measurable |
| with payload model | differences in the vehicle free responses |
| | Measurable differences in vehicle response |
| | due to different types of payloads |
| ½ car model | Moment of inertia in pitch has dominant |
| sensitivity and | influence on vehicle's natural frequencies |
| Monte Carlo analysis | Suspension stiffness is a significant contributor |
| | to vehicle dynamic response |
| ½ car model | One way to identify increased weight in vehicle |
| simulation results | is to measure the peak-to-peak period of |
| | bouncing or pitching motion after the vehicle |
| | traverses a speed bump |
| | Load classification could be determined by |
| | examining the frequency and amplitude change |
| | of a vehicle response after it traverses speed |
| | bump |
| ½ car model | Change in peak-to-peak frequency validated on |
| experimental | a Chevy Astro van |
| validation | |
| Cleat configuration | Cleat layout excites mode at 1-2 Hz and has |
| experiments | ability to measure the free response of the |
| | vehicle to detect an added load. |
| Vehicle database | CARSP database provides information on |
| collection | vehicle mass, weight distribution, and wheel |
| | base for 20K cars |
| | Data on suspension stiffness is not presently |
| | available |
| Vehicle parameter | Methods developed to estimate missing vehicle |
| estimation to | parameters for ½ car model |
| supplement established | |
| databases | |
| Sensitivity analysis on | Identifying vehicle model and wheelbase can be |
| vehicle parameters | used to determine vehicle's curb weight |

Tasks Performed: Door panel vibration sensing

| Tasks | Results/Notes |
|---|---|
| Modeling of | Model of panel exhibits approximately the same |
| car door panel | behavior as was observed experimentally |
| Modal impact | Modal impact test of unloaded car door and |
| testing of car | additional of several internal payload |
| door panel | configurations |
| Vibration test of | May be difficult to model |
| car door during | |
| cleat pass-over | |
| Analysis of | Shift in resonant frequency and vibration |
| experimental results | amplitude identified |
| | Use of amplitude integration can reveal possible |
| | loading of car door |

Tasks Performed: Acoustic sensing

| Tasks | Results/Notes |
|---|---|
| Literature review of | Previous research focused on force on axle |
| previous tire acoustic | due to acoustical mode |
| mode research | Phenomenological effects of tire loading/ |
| Vibration test of tire | Wavenumber decomposition of tire vibration |
| sidewall | allows for visualization of wavespeeds, |
| | cut-on frequencies |
| | Increased carcass vibration wavespeed due |
| | to increased tire inflation pressure |
| | Tire acoustical mode visible; more defined at |
| | higher frequencies |
| Acoustic | Measurements around tire along with axle, |
| measurements of | tire tread |
| radiation from tire | Differences in air-filled and insulation-filled |
| | tires |
| | Spike in acoustical signature due to tire |
| | acoustical mode seen in air-filled tire but not |
| | in insulation-filled tire |
| | Variation of spike amplitude corresponds to |
| | expected shape of acoustical mode |
| Drop tests | Distinct differences in signature of air-filled |
| | and insulation-filled tires when dropped. |
| | Drop tests more accurately simulate force of |
| | pavement on rolling tire. |

### Sensing and Analysis of Vehicle Visual Appearance (e.g., Video Analysis)

Video analysis and intelligent surveillance systems as used in some embodiments of the present disclosure are useful for the purposes of law enforcement and security. Embodiments of these systems can detect irregularities and/or anomalies in vehicles that can indicate the presence of hidden or disguised materials, such as drugs, hidden persons, explosives, and general contraband. Irregularities in vehicle orientation (e.g., whether the vehicle is leaning nose up/down and/or to one side due to a heavy load), vehicle travel direction, vehicle speed, etc.) may be detected by one or more embodiments.

Deployment of autonomous visual surveillance systems to monitor vehicles is especially attractive due to the large, and increasing, volume of traffic. The role of video analysis in various embodiments may be broadly classified under two categories:
1. Estimation of vehicle physical parameters including dimensions, make/model, and occupancy. This information can be used by other sensors to determine the expected ranges of “normal” measurements under such conditions. The video system can also be used as a trigger for potentially all the sensors, activating whenever an approaching vehicle is detected.
2. Behavioral analysis of the vehicle to detect anomalies in the appearance and motion.

This information can be directly used to classify a vehicle as suspicious if the system observes a behavior that matches one of the templates associated with anomalous behavior. It is also possible to further categorize this information such that occurrence of certain severely anomalous patterns would be sufficient to flag the vehicle for further screening. In other cases, the anomalies could be evaluated in the context of possible anomalous signatures from other sensors.

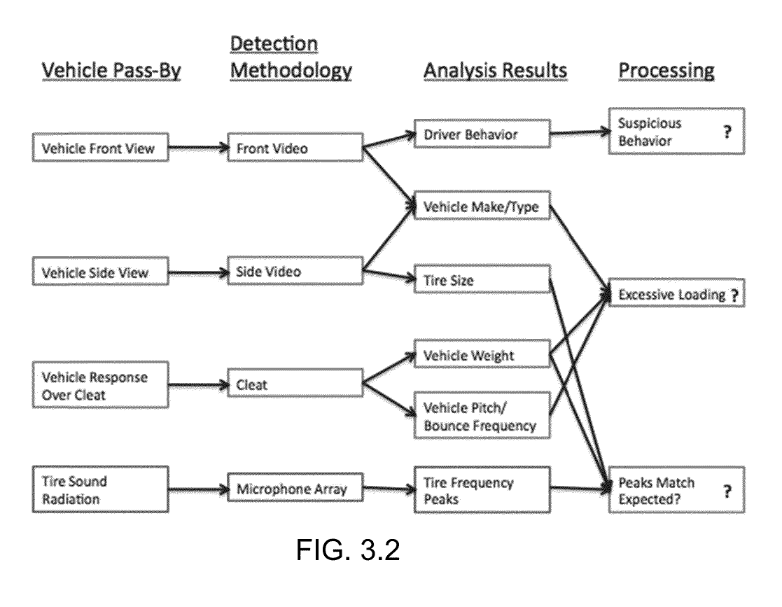

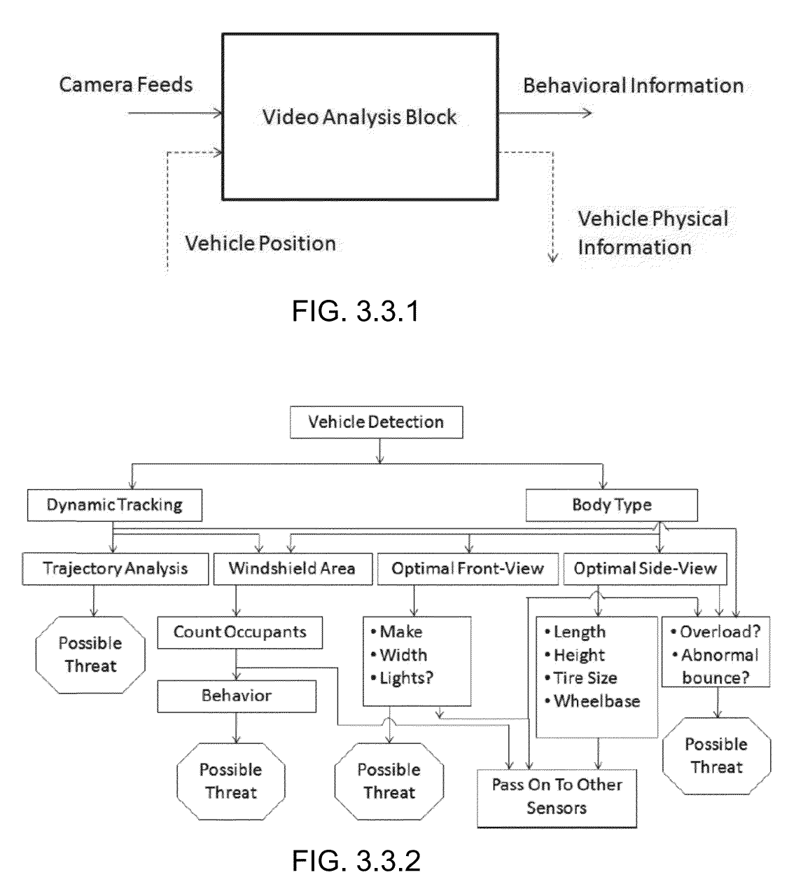

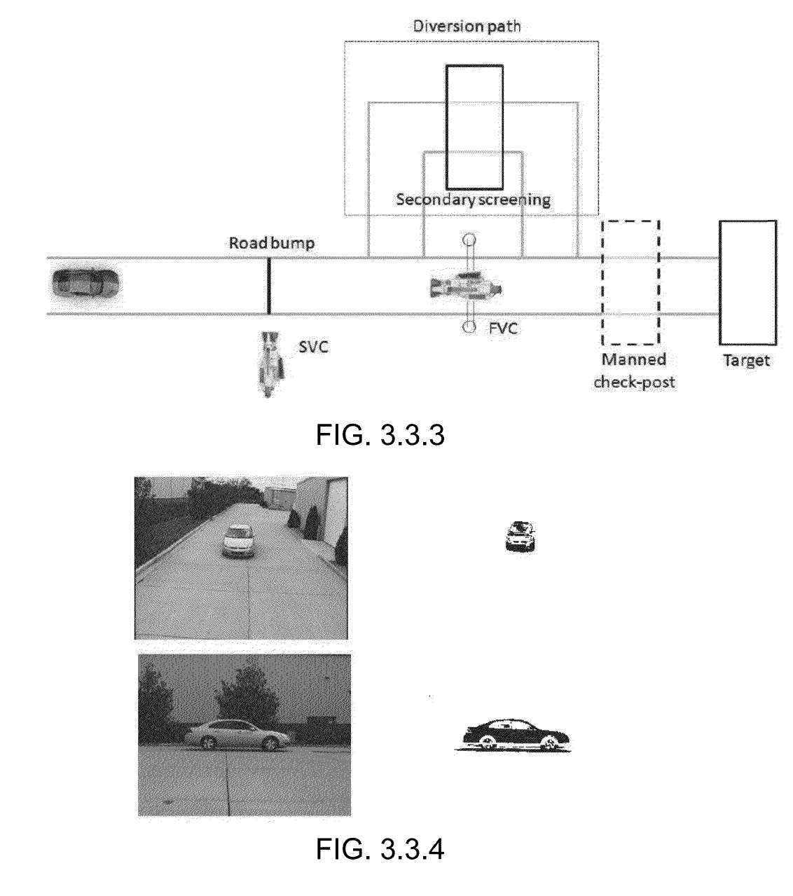

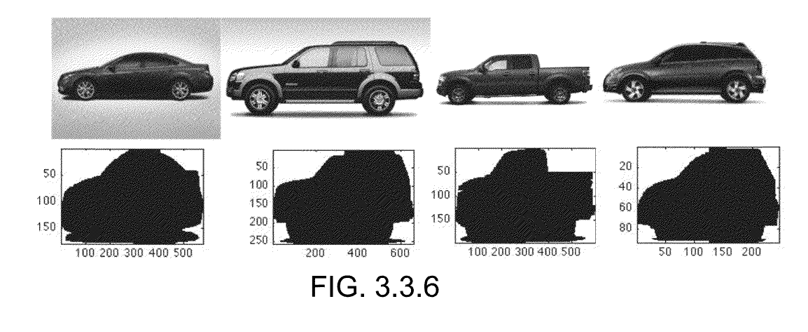

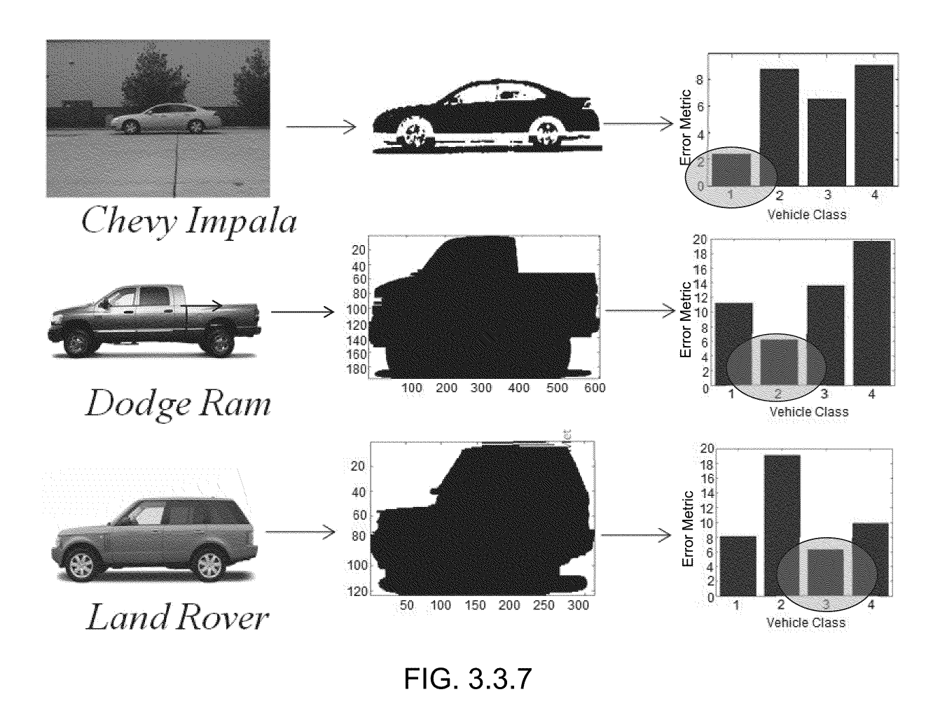

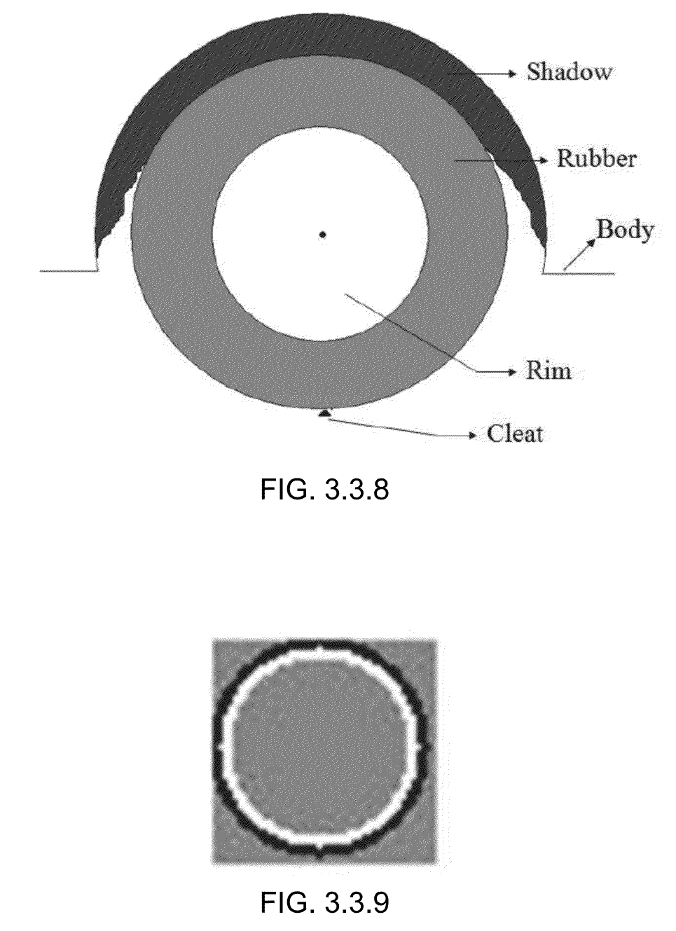

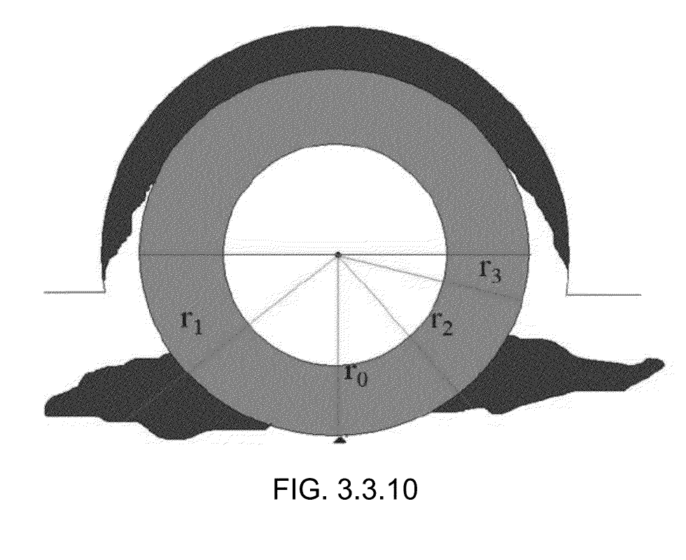

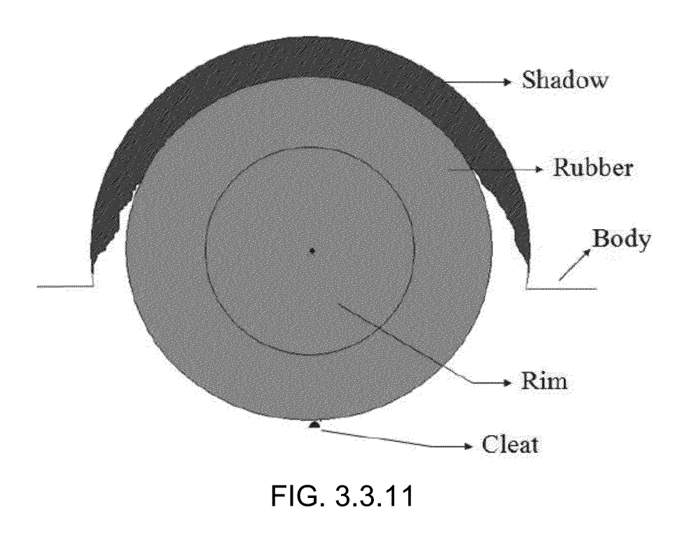

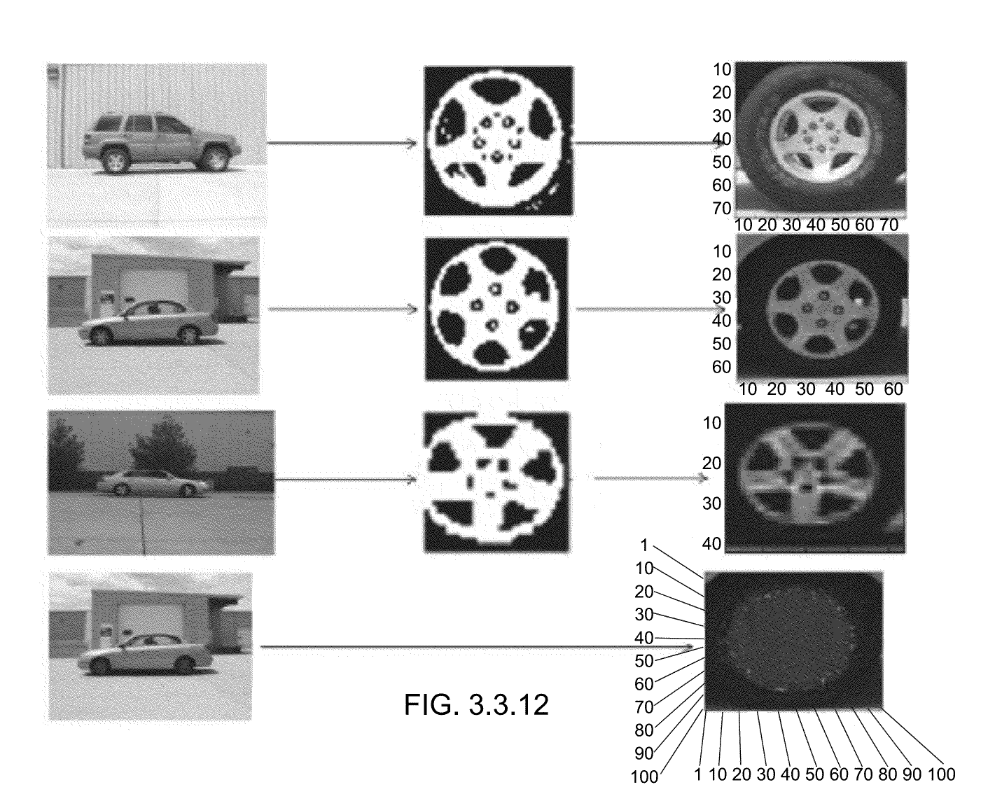

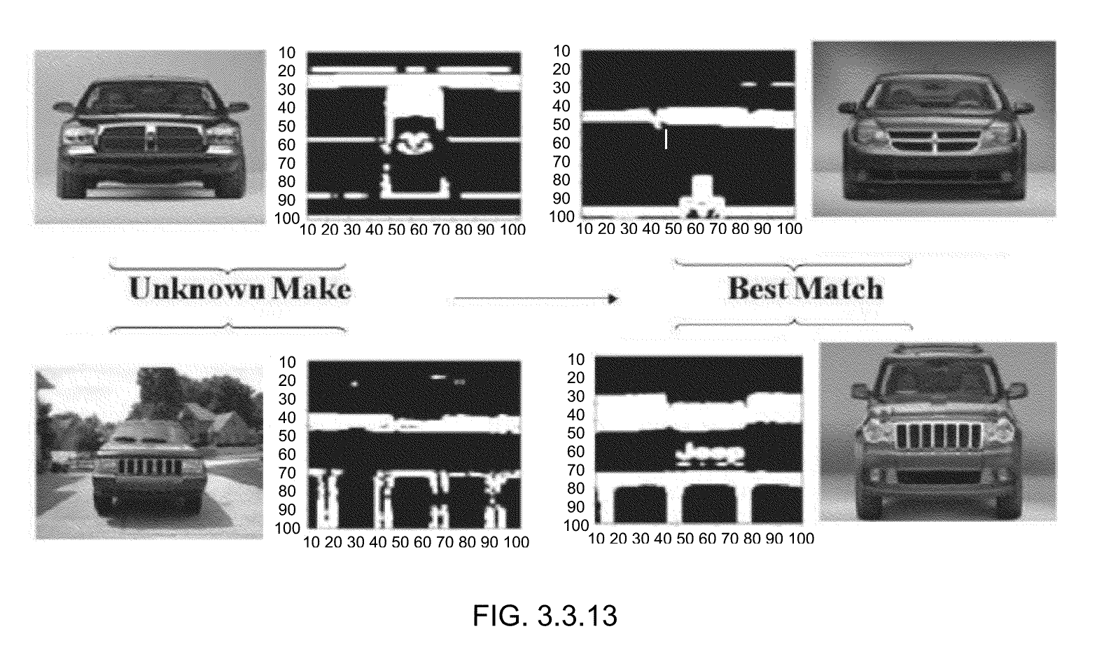

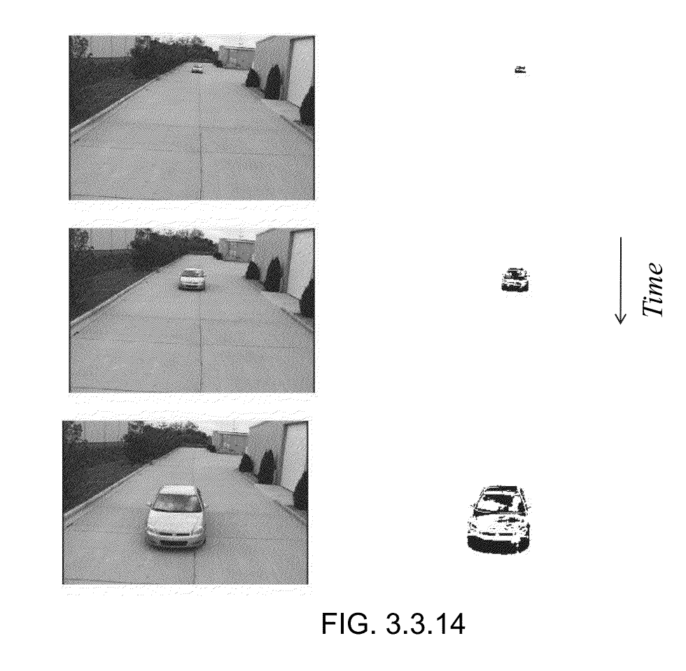

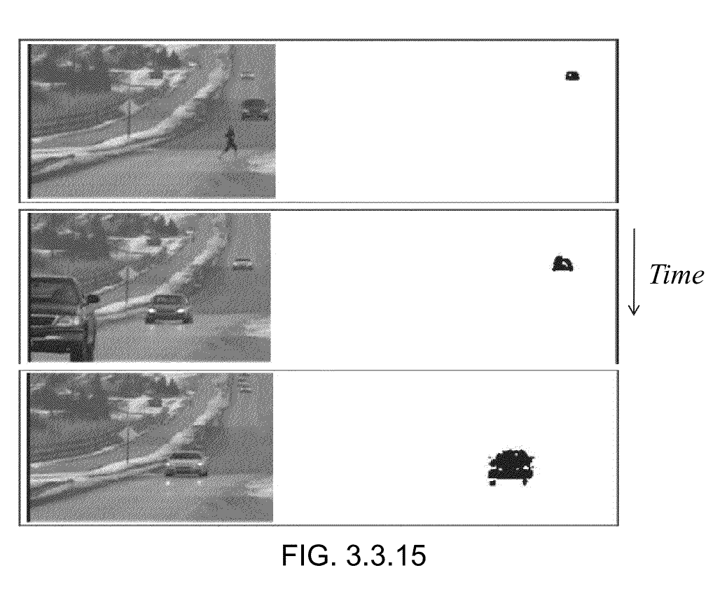

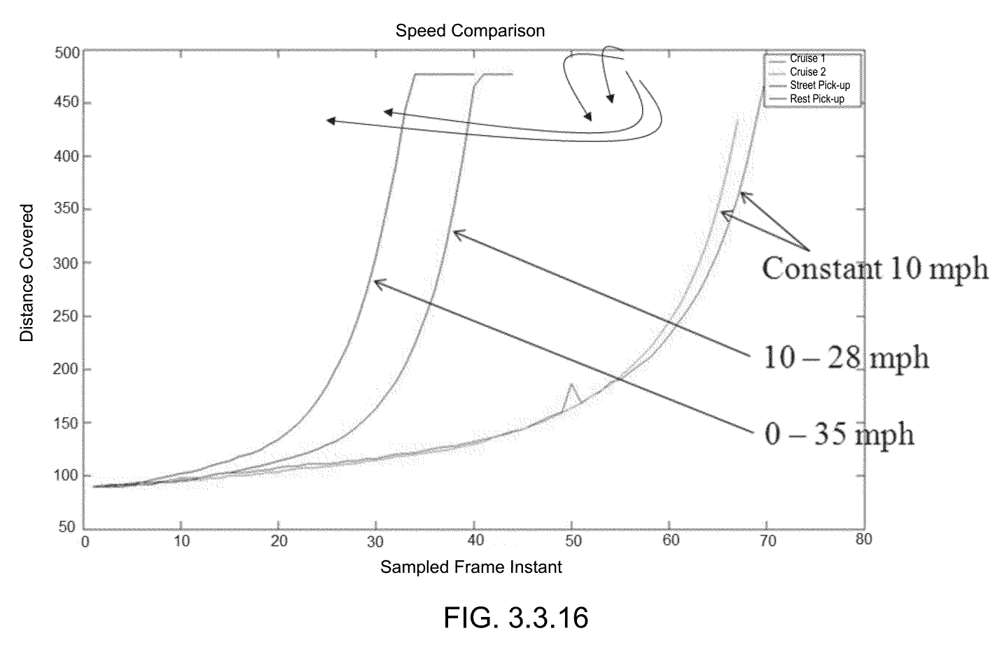

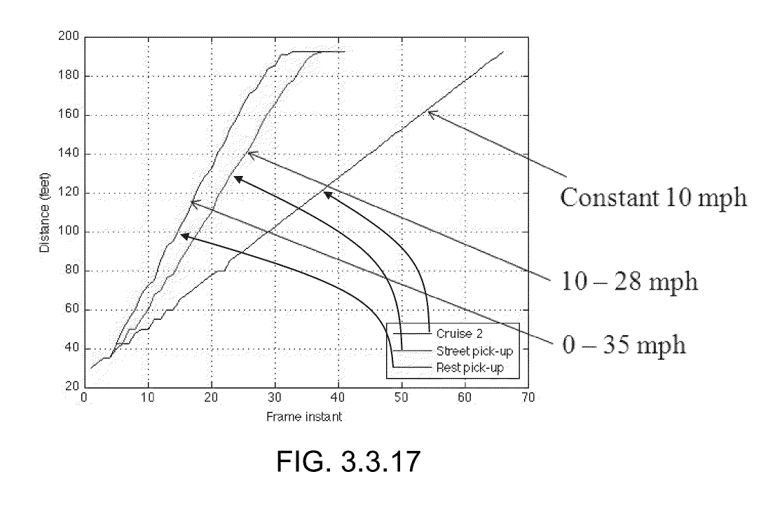

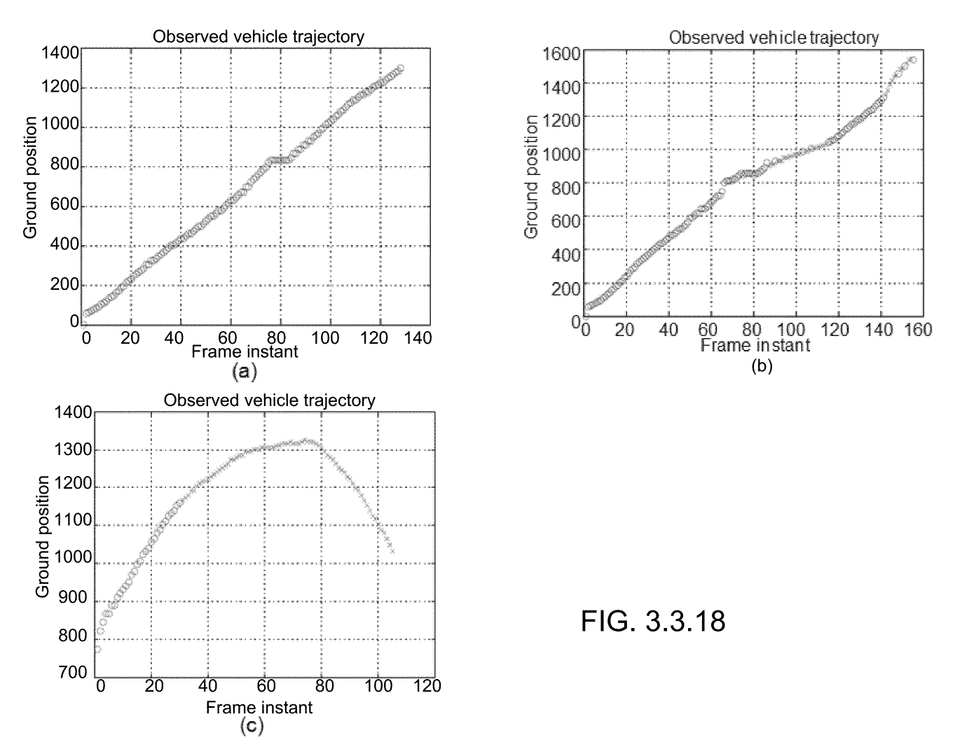

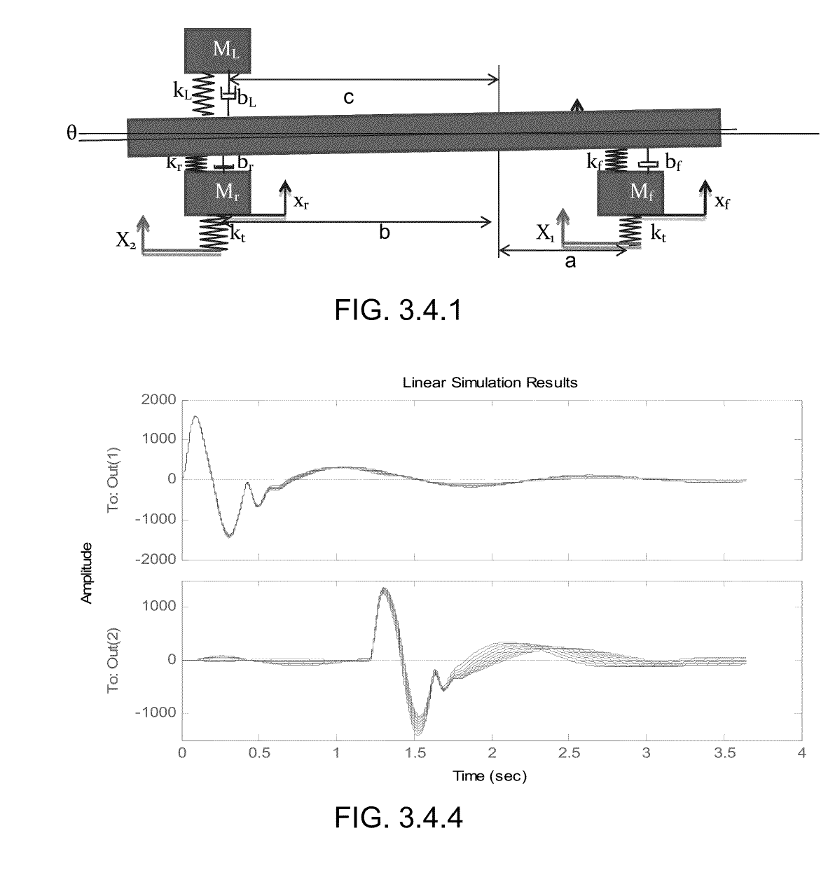

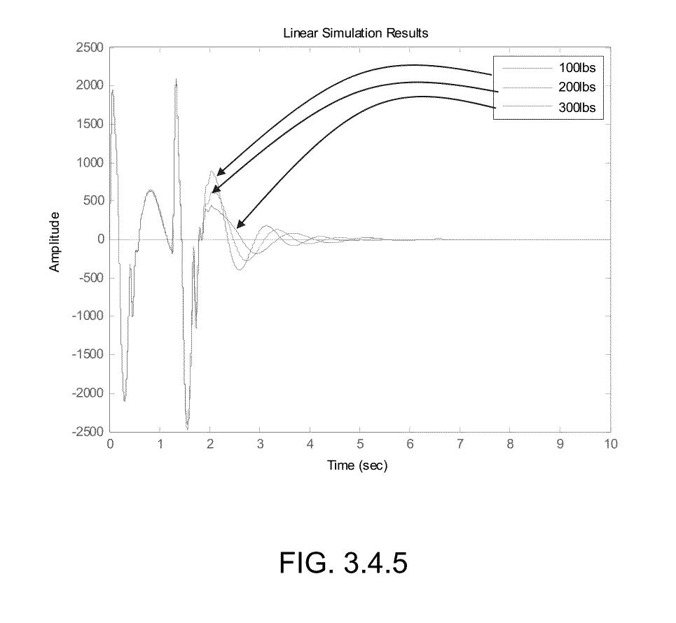

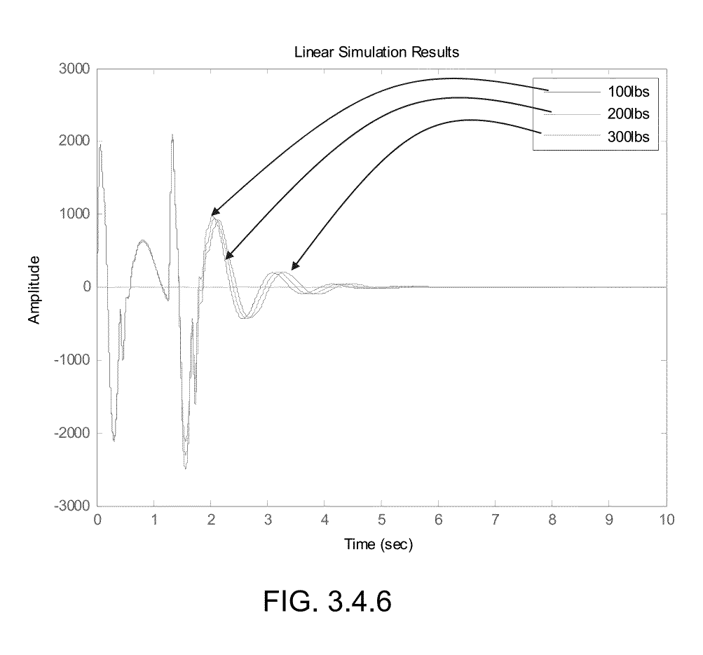

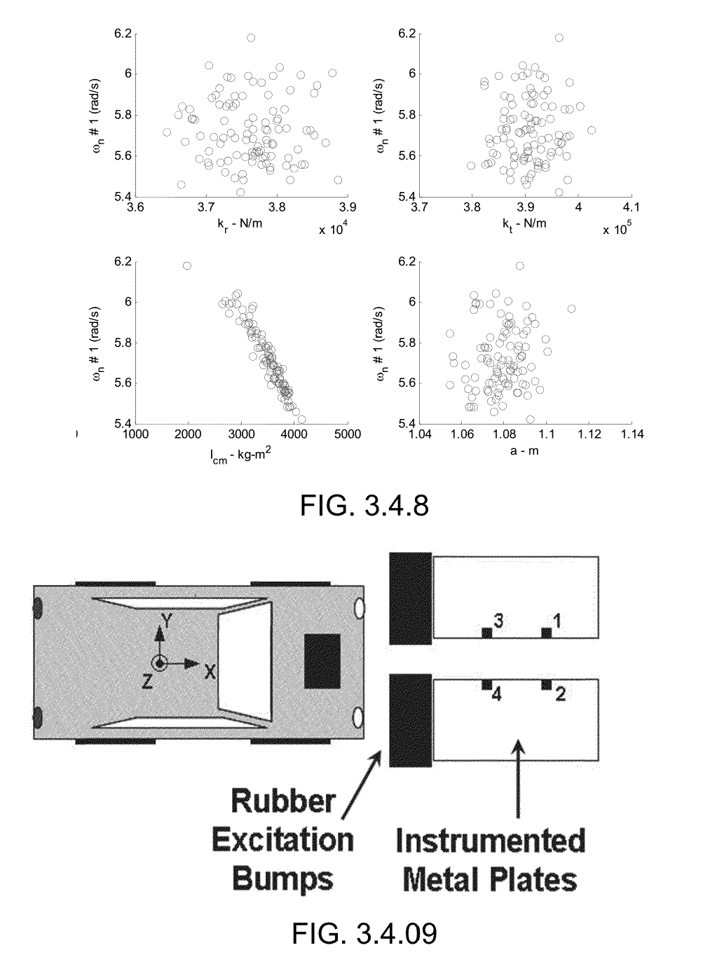

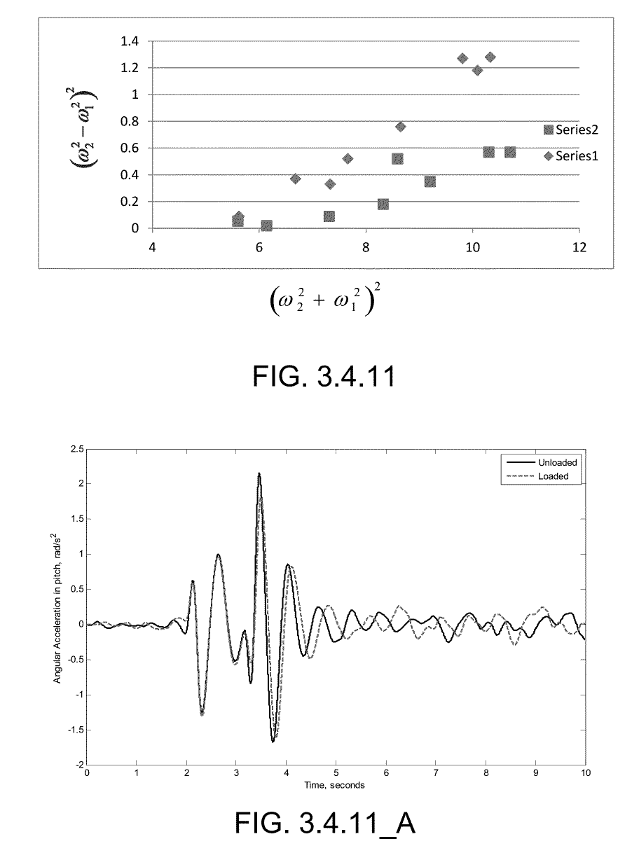

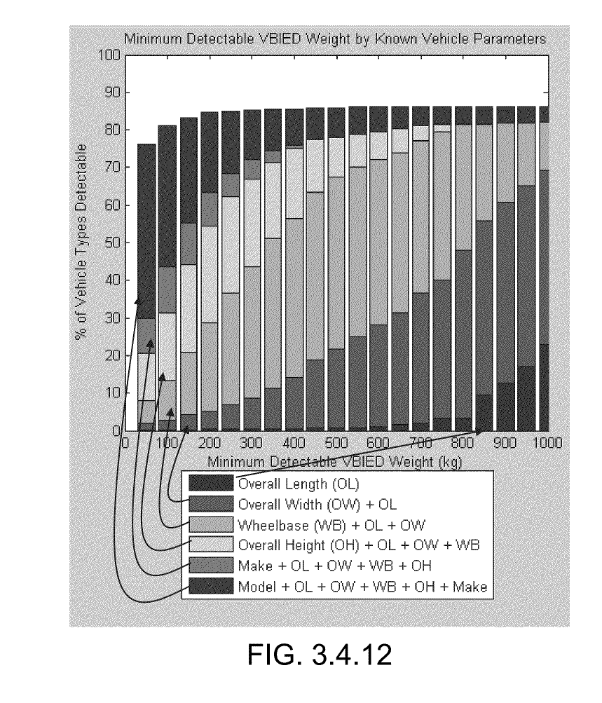

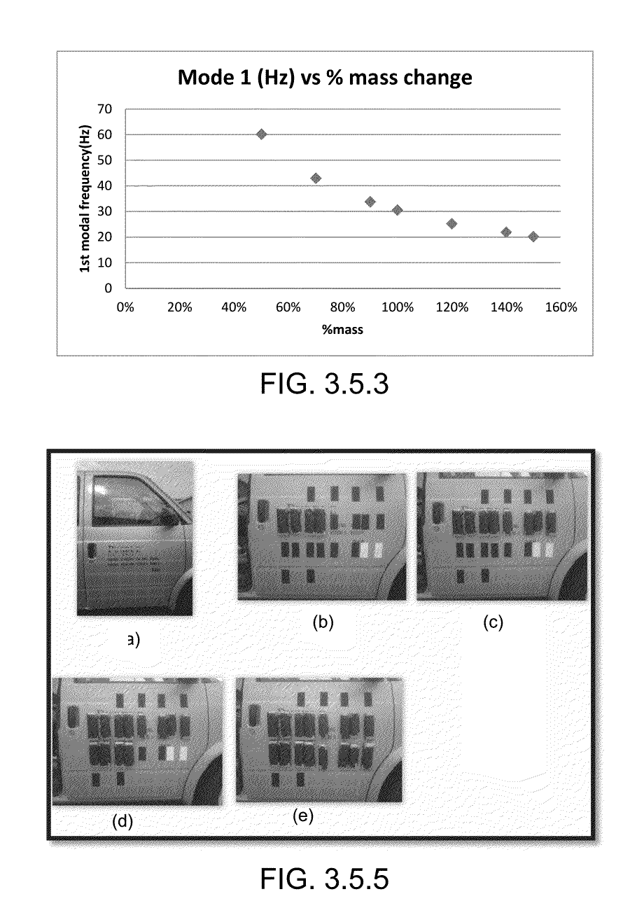

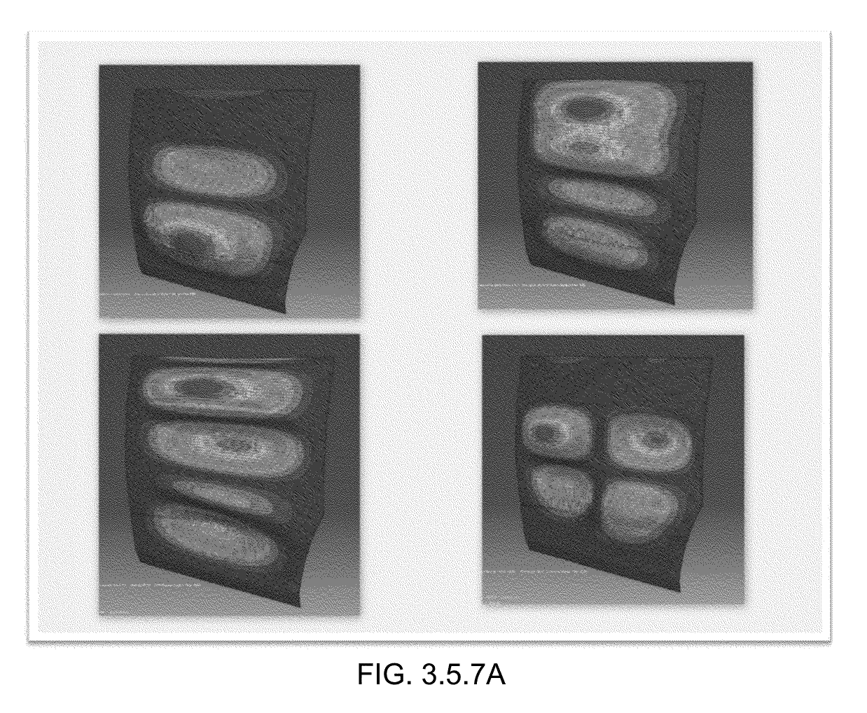

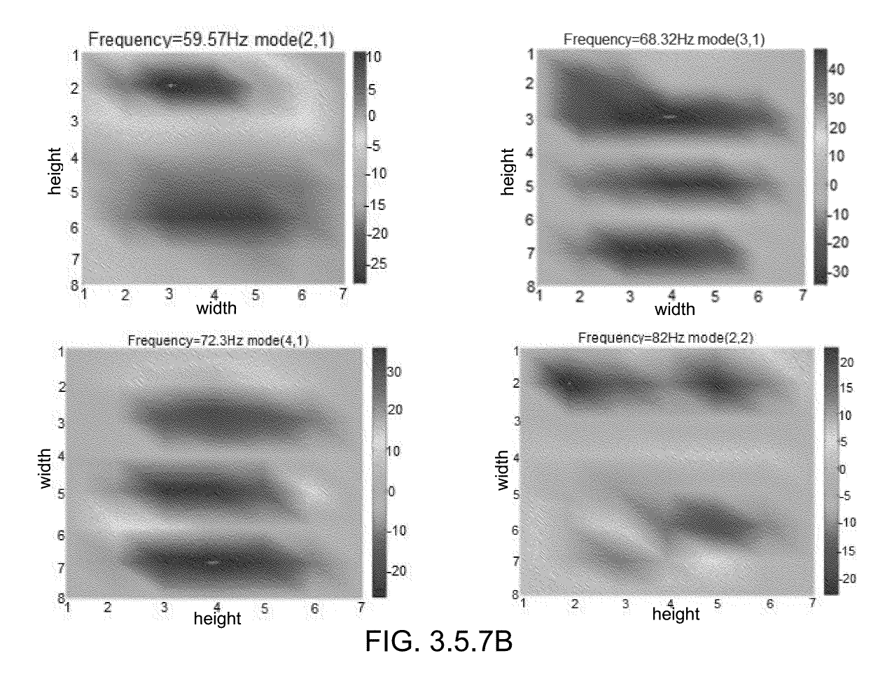

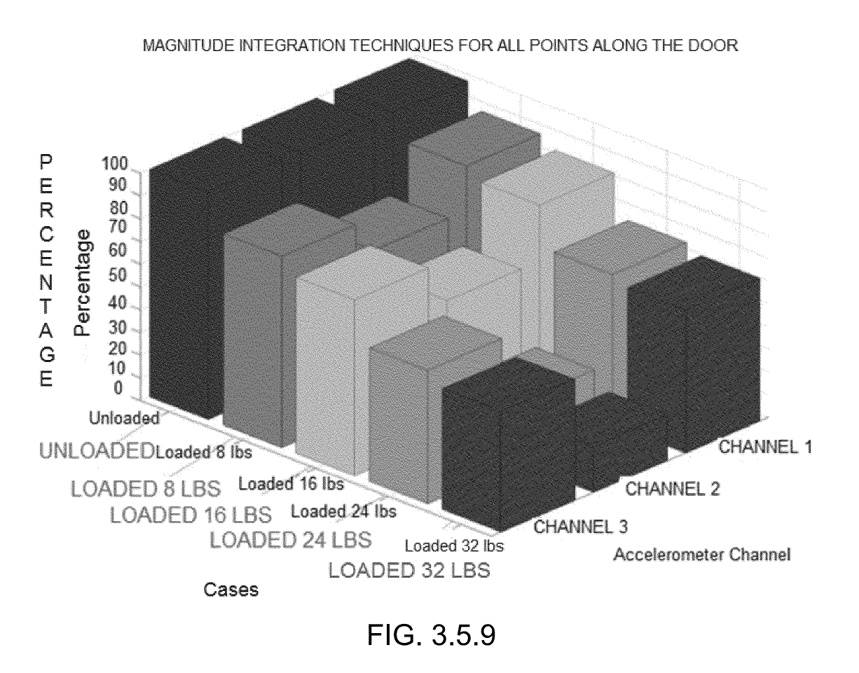

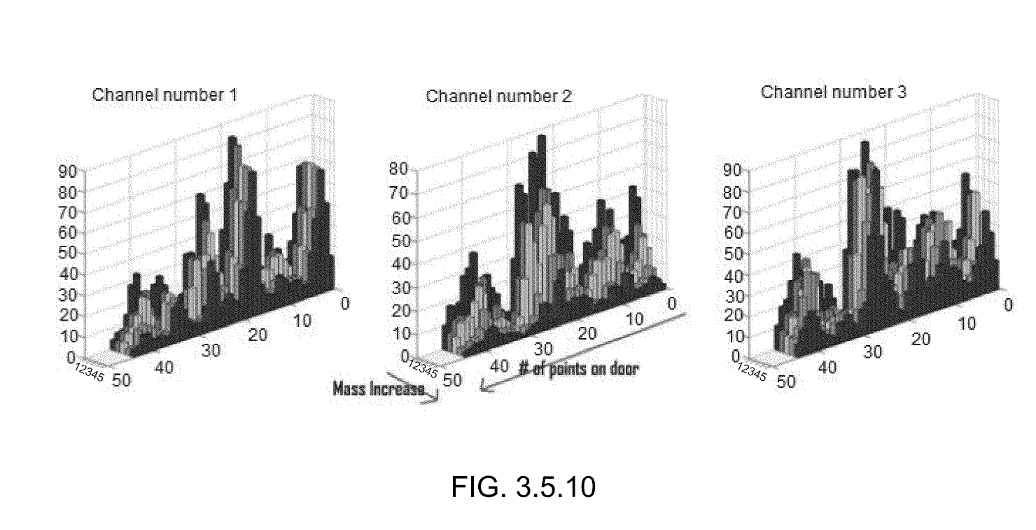

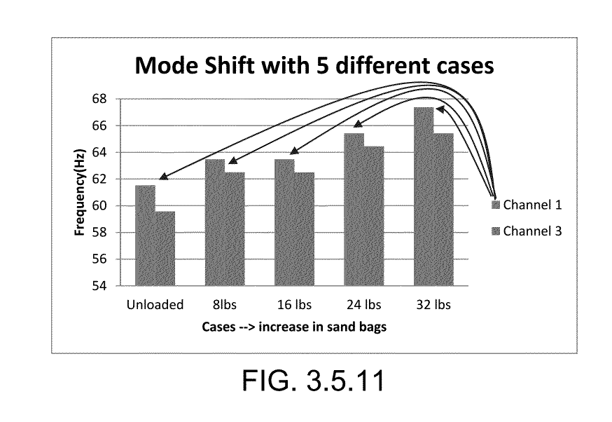

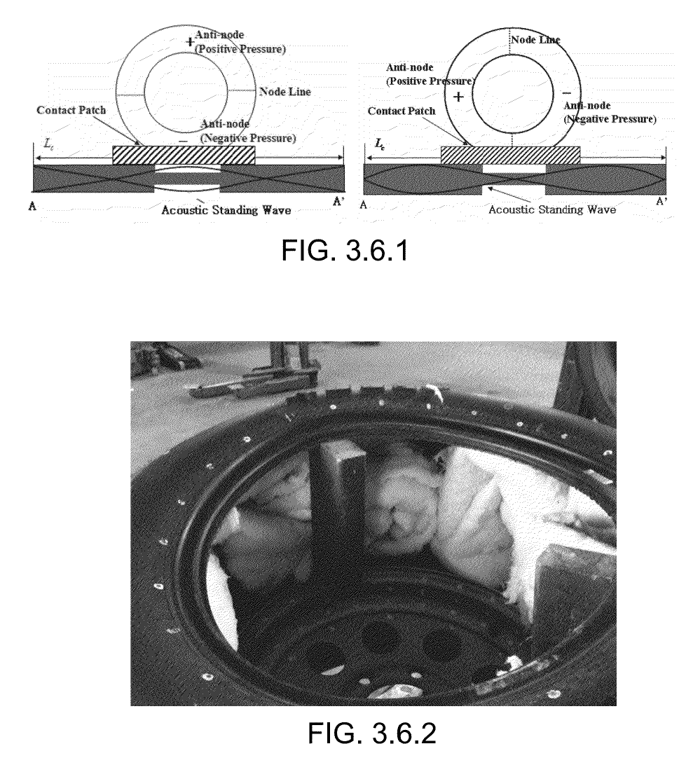

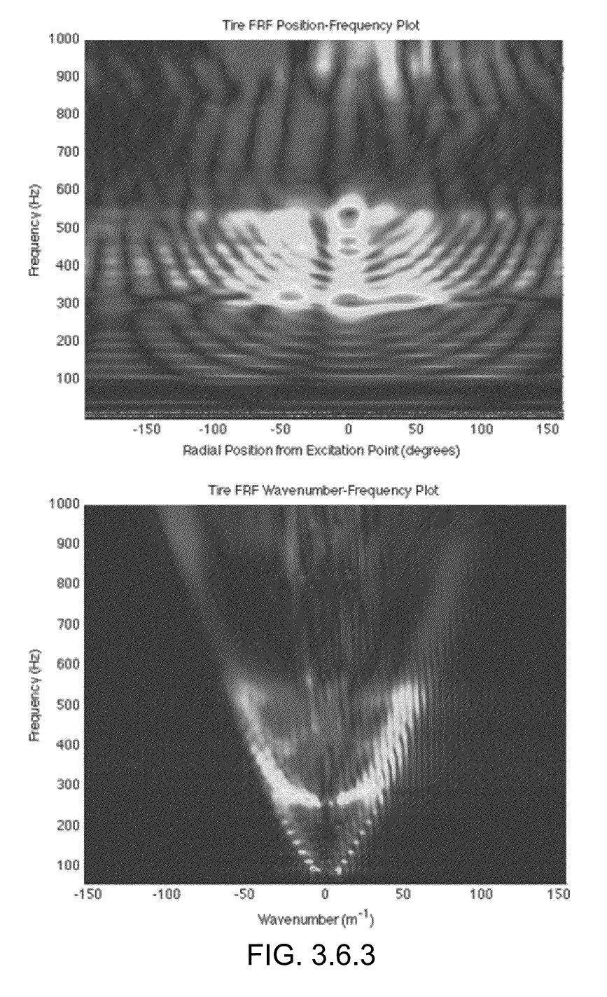

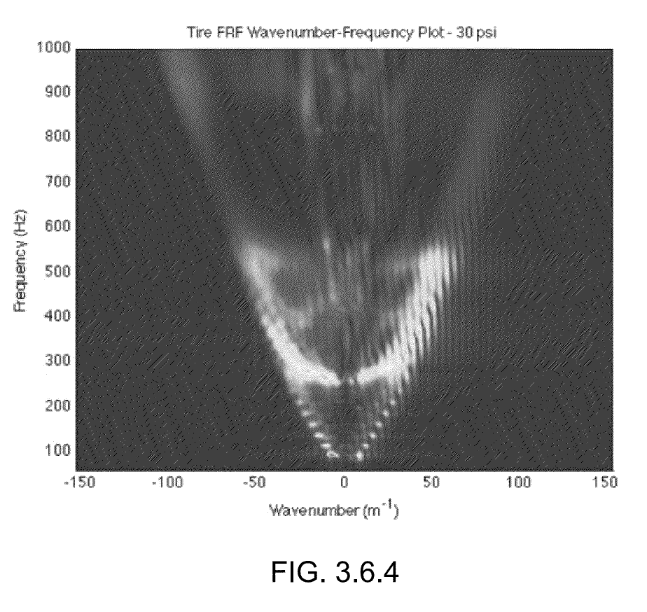

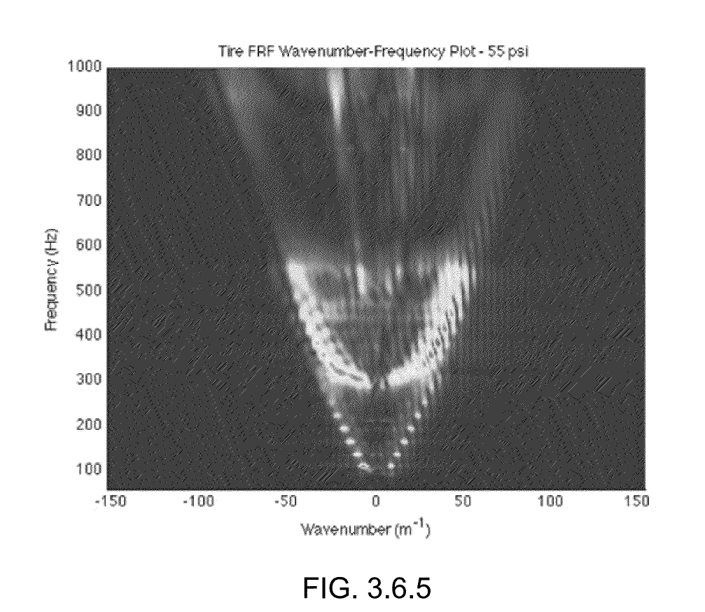

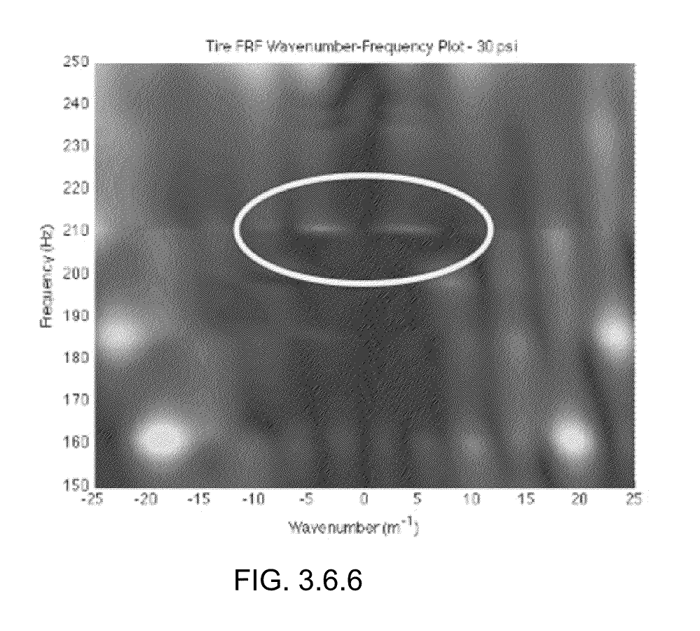

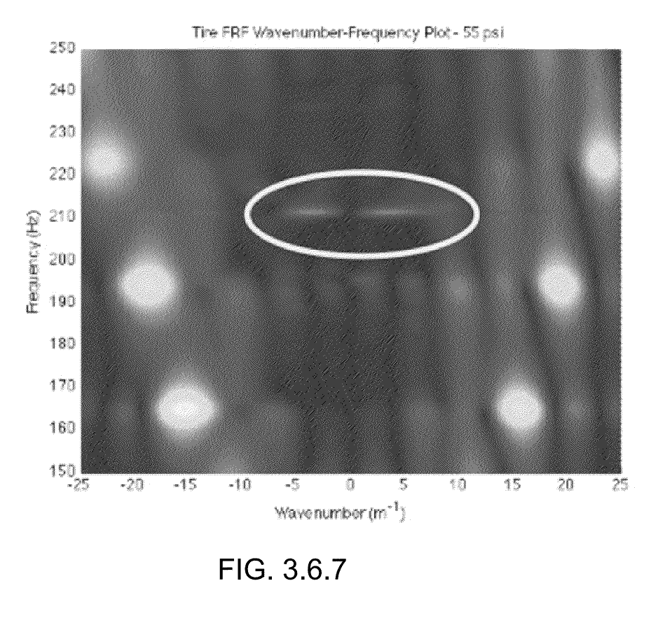

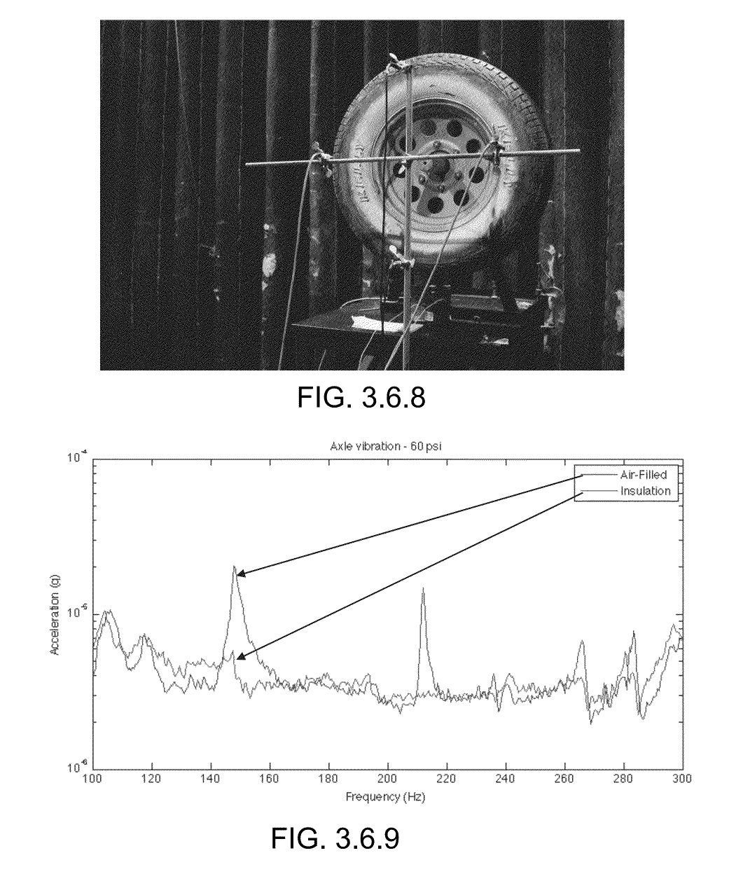

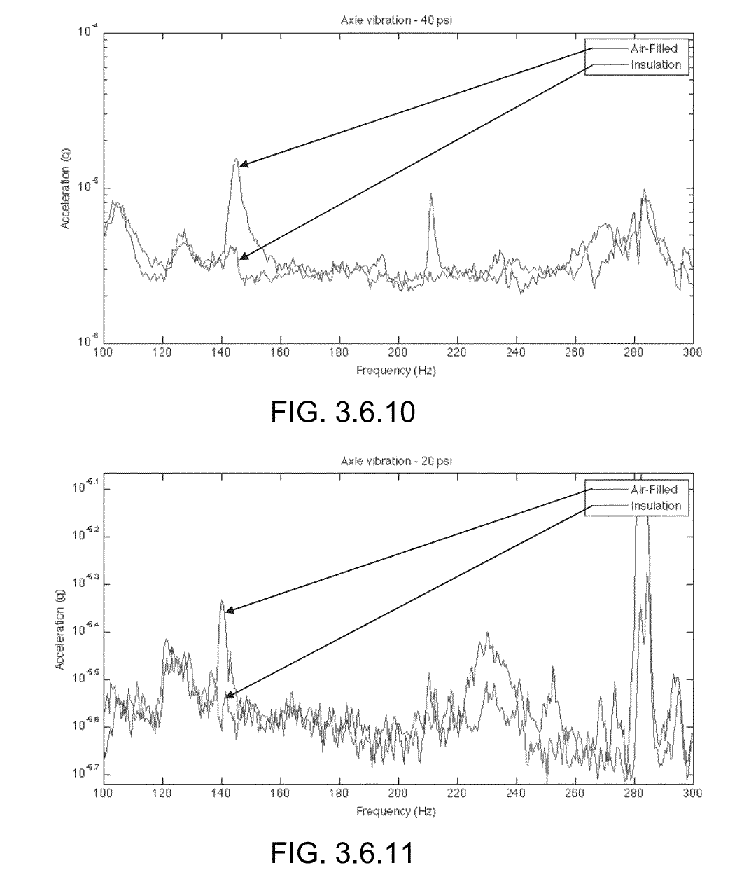

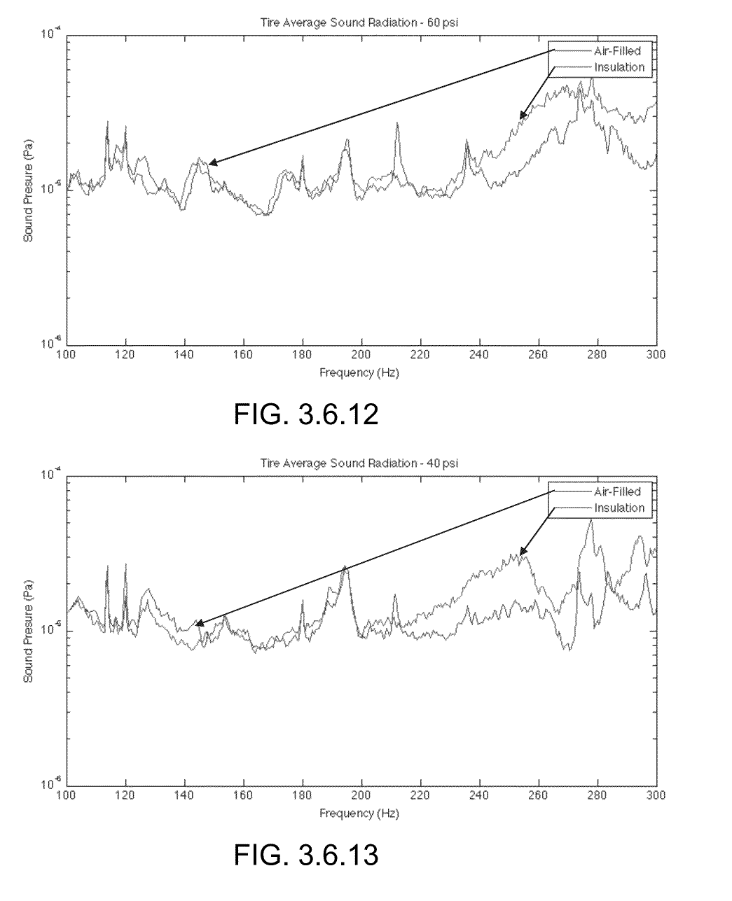

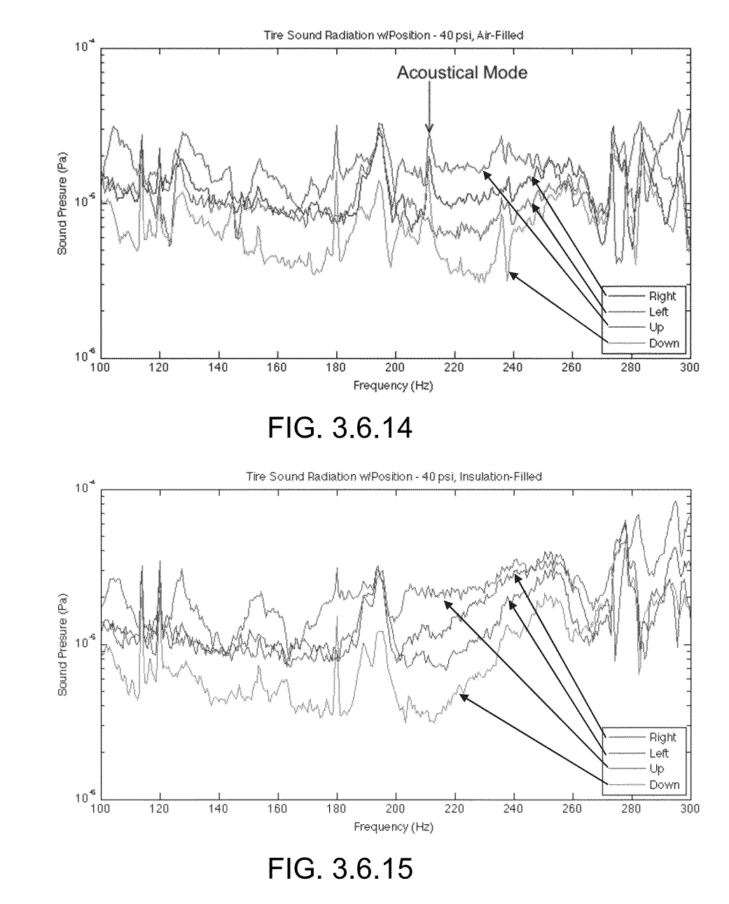

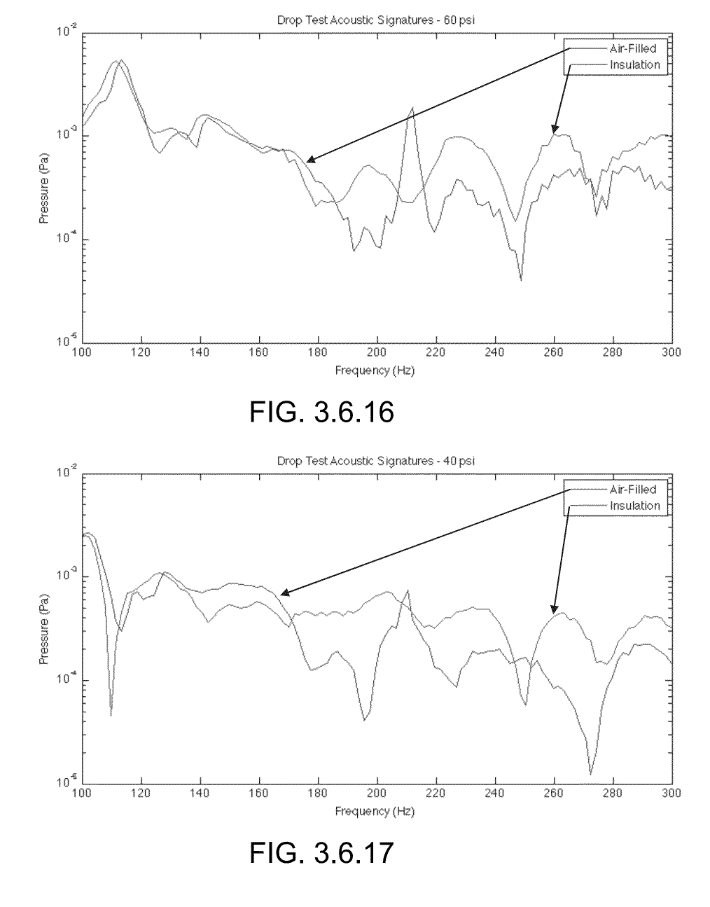

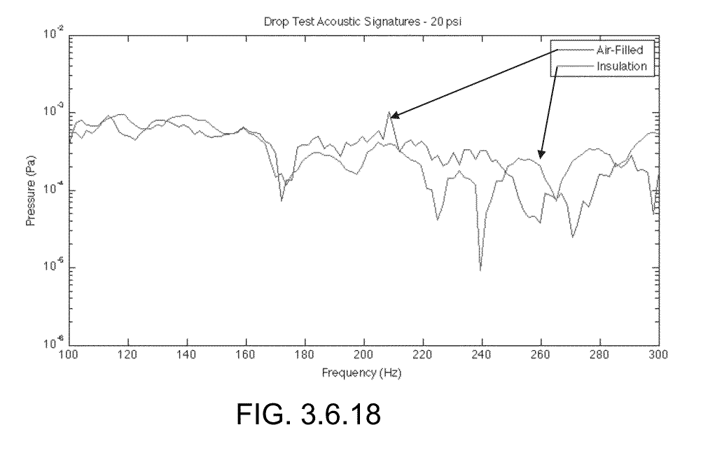

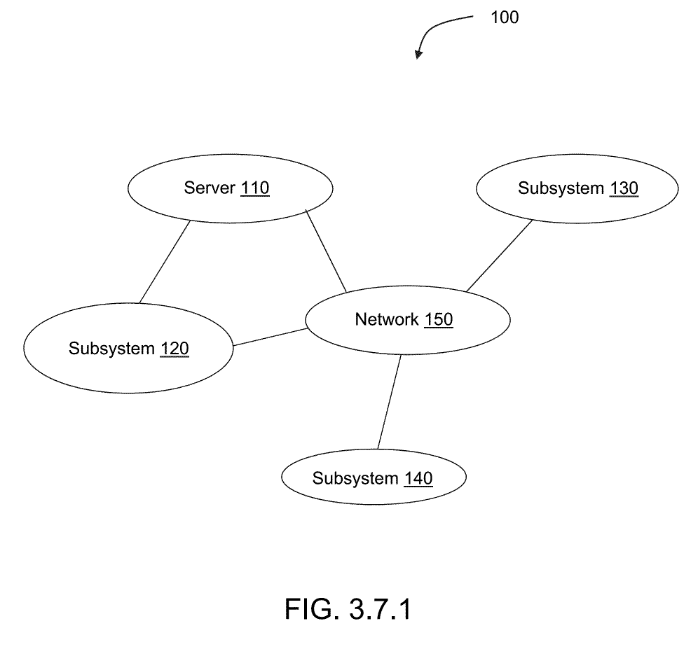

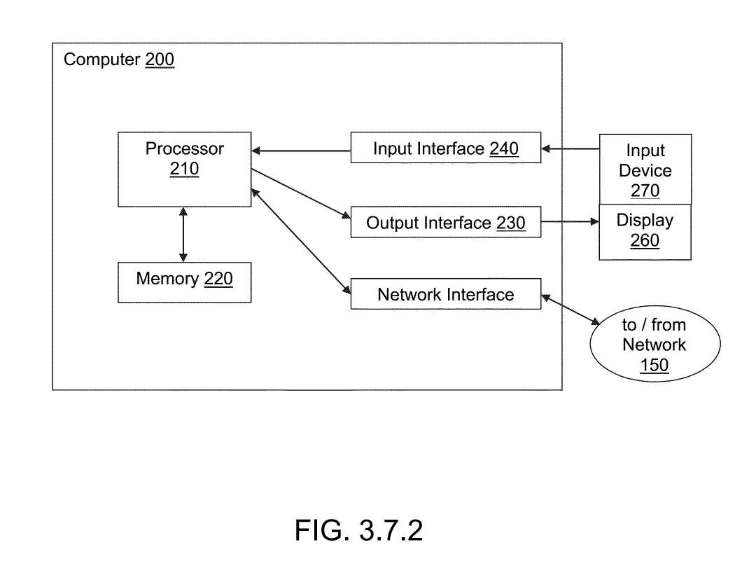

It should be pointed out that the above classification is not exclusive. That is, certain measurements (for example velocity) could be placed under either, or both, categories. The operation of the video analysis system according to one embodiment of the present disclosure is depicted in FIG. 3.3.1. A block diagram of a video analysis system according to one embodiment of the present disclosure is depicted in FIG. 3.2. The solid arrows represent the primary inputs and outputs of the block in the sense that such outputs are directly used by a decision unit to detect an anomaly. The dashed arrows represent auxiliary information shared between the video system and other sensors. The importance of this information sharing can range from marginal to critical, and has been described in greater detail in appropriate sections.

Each of these goals is accomplished by performing a number of smaller tasks. These smaller tasks, and their interdependence, are graphically illustrated in FIG. 3.3.2. Detailed methods for the individual detection tasks are provided in the following sections.

It is natural that many of the tasks above cannot be achieved when looking at a vehicle from a single view. Some embodiments include at least two video cameras providing two different views, e.g., front and side views (as mentioned in FIG. 3.3.2). The position of the two cameras with respect to the check point is according to one embodiment shown in FIG. 3.3.3. Note that the figure is not drawn to scale and the front view camera (FVC) is placed at a certain height above the ground.

### Robust Vehicle Detection

In at least one embodiment vehicle detection is performed by a background subtraction technique to segment the foreground object (vehicle) from the background. This analysis may be used in conjunction with various cameras, such as a front view camera (FVC) and/or a side view camera (SVC): The output of this process is a binary image in which each pixel is classified as belonging to the foreground or the background, for that frame. Let C={c(i,j)} and B={b(i,j)} represent the current frame and the background model at any time instant. The indices (i,j) are used to specify the spatial location in the image. Then, a pixel at (i,j) is classified as a foreground pixel if |c(i,j)−b(i,j)|>threshold.

The background model B and the decision threshold may be updated during the detection process in some embodiments to account for changes. However, these steps can cause a decline in the processing speed. We note that in at least one embodiment, a background model update is not required in every frame. On the other hand, since the system is likely to be deployed in outdoor scenarios, the background would likely have considerable noise. Thus, alternate embodiments include a method for background subtraction in which each pixel of the current frame is compared with a neighborhood of the background model. This change can account for small camera motion as well as undesirable motion in the background (like tree leaves). In these embodiments, a model update is applied only when a change of illumination is detected.

An example output of the object detection technique, applied to vehicles, is presented in FIG. 3.3.4 (Top: FVC, Bottom: SVC).

The above-described technique may be referred to as MABS (Multi-Agent Based Simulation). Alternate embodiments can use other algorithms for object detection, e.g., background subtraction with adaptive threshold (ATBS) and background subtraction with Gaussian mixture models (GMM). The three techniques (MABS, ATBS, and GMM) were applied on video sequences to detect humans. While all three techniques detected humans, the MABS method generated smallest number of “false-positives” due to background clutter. The running time of these methods was computed for five different sequences. These are provided in Table 3.3.5, and the speed advantage of the MABS algorithm is evident.

TABLE 3.3.5
Running time of object detection algorithms (in seconds).

| Sequence | Frames | GMM | ATBS | MABS |
|---|---|---|---|---|
| 1 | 100 | 1020 | 214 | 102 |
| 2 | 100 | 994 | 213 | 104 |
| 3 | 180 | 1910 | 374 | 225 |
| 4 | 270 | 2150 | 446 | 224 |
| 5 | 210 | 2092 | 443 | 193 |

### Vehicle Body-Type Analysis

Make and model identification (such as by using the SVC or other cameras) as used in some embodiments is useful to the vehicle dynamic analysis. To assess vehicle make and model, a silhouette-matching based approach is adopted in some embodiments to classify vehicles into one of multiple types. In one embodiment, four types are used—sedans, light trucks, sport utility vehicles and hatchbacks. Toward this end, a “clean” image of a representative vehicle is chosen for each class, and processed to obtain a silhouette. High quality images with plain background have advantages by assisting to ensure a sharp template is available for comparison even when the test images are of low quality. However, lower quality images or images with backgrounds that are not plain are used in alternate embodiments. The selected vehicles and their templates are shown in FIG. 3.3.6.

When deciding on the type of a test vehicle, one or more of the following steps may be followed:
The output of object detection (SVC) is cropped to the smallest size (say, h×w) such that all the foreground pixels are still retained.
Each class template is scaled to the same size (h×w). Note that the templates are already appropriately cropped.
The cropped test image and the scaled templates are subtracted and a sum of absolute difference (SAD) is computed for each class.
The class with the lowest SAD determines the vehicle's type.

The results of applying the above methods on some test vehicles are shown in FIG. 3.3.7.

Tire Size and/or Wheelbase Analysis

Vehicle tire size can be used in some embodiments to detect anomalous tire conditions (such as by using the SVC or other cameras), and may be used to assist the acoustic determination of anomalous tire conditions. The task of accurate estimation of a vehicle's tire size can be made difficult by the lack of information about the tire's position, and the variation in shapes of the hub. However, in most vehicles with shiny wheels or hub caps, the tire can be modeled as overlapping dark and light disks as shown in FIG. 3.3.8. For a moving vehicle, the pattern of the hub is frequently blurred, but there exists a contrast between the dark rubber and the hub.

In order to estimate the tire size, one needs a feature that would remain invariant for a large fraction of vehicles observed. It is noted that the circular edge between the dark rubber and the lighter hub is a visible feature and remains invariant to the actual design of the hub. This edge is modeled as a pair of concentric circles of chosen radius as shown in FIG. 3.3.9.

An estimate of the visible wheel size is obtained by superimposing the above model over various positions of the vehicle and computing the absolute difference. Note that the process is repeated for different wheel radii and the region shown in gray is not considered in the difference operation. The size and position which result in the lowest error are chosen as the estimate of wheel radius (r_{w}) and the tire center position (xₜ, yₜ), respectively.

From the estimated tire center, straight lines can be dropped toward the road surface till the rubber-road edge (or another edge with similar features) is reached. This is shown in FIG. 3.3.10. The smallest of these lines' lengths, greater than r_{w}, gives the overall tire size estimate. Thus,

rₜᵢᵣₑ=min{r₀,r₁, . . . ,rₙ/rᵢ>re}.

For the situation depicted in FIG. 3.3.11, there is no distinct rubber-wheel edge. However, if the horizontal location of the tire center is available, an estimate of the tire size can be obtained by using a dark disk as the model. The process is similar to that used for locating the wheel-rubber edge in the previous case, except that only the radius and the vertical location are changed during the search.

Some examples from applying the above methods to test vehicles are shown in FIG. 3.3.12. Note that in these examples, the horizontal position of the tire center (or equivalently, the position of the cleat) was specified.

### Vehicle Make/Model Recognition

Some embodiments utilize Make Model Recognition (MMR) and can operate by using the rear view of a vehicle wherein the location of the license plate and the text of the Make/Model information can be utilized. Other embodiments use the front view and assume the presence of a license plate which facilitates selection of a region of interest (ROI).

Still further embodiments perform an automatic ROI selection on the test vehicles such that the front grille of the vehicle is contained in the frame. Instead of choosing a reference object (like license plate), have regions selected based on the vehicle′ body type (an information reliably available from the SVC). Once the ROI is selected, we compute the gradient of edges at every point in the region is computed and a histogram is generated. Note that the histograms for the representative vehicles for each Make being investigated can be pre-computed. The histogram of the test vehicle is compared with each template histogram and the correlation is computed. The Make resulting in the highest correlation is typically determined to be the vehicle's Make.

FIG. 3.3.13 shows two examples of successful Make determination where the left-most image is the test vehicle while the right-most image is the representative vehicle for the Make. The tests were conducted using a small set of Makes. It was also ensured that a test vehicle image was not one of the representative images for the set.

### Vehicle Trajectory Analysis

Challenges for any tracking system include changes in the object/surroundings over time, partial/total occlusion by other objects, and high computational complexity. Tracking across video frames can be achieved by repeated moving object detection or by a feature-based spatial search for the object. Both approaches may be used (either together or separately) in some embodiments for vehicle tracking. It should be noted that many methods can be used to overcome the challenges of robust tracking and these can be selected based on the application.

The object detection based tracking approach is frequently used to locate the foreground pixels (blob) corresponding to the vehicle for every frame, while a particle filtering method may be used for feature-based tracking using, for example, color and edge orientation as object features. The results of detection based tracking on test vehicles are shown in FIGS. 3.3.14 and 3.3.15.

Particle filter is a probabilistic technique for state estimation based on state transition and observation models used in some embodiments. The “state” can be defined as the set consisting of one or more of position, velocity and the size of an object. The state transition model predicts the state of the object for the next time instant, based on the previous states and observations, while the observation model verifies/corrects the prediction using the current observation (which consists of the chosen features). It should be noted that tracking a vehicle (such as by using the FVC or other cameras) frequently includes the size in the state as the blob corresponding to the vehicle changes considerably over time.

Once an appropriate method for vehicle tracking has been selected for the particular situation and the output obtained, one can designate a point (or a set of points) as representative of the vehicle and record its position for every frame to get the trajectory. In some embodiments a point which is located in the middle of the vehicle blob, horizontally, and is close to the lower frame boundary is selected. FIG. 3.3.16 shows an example of recorded trajectories (in image co-ordinates) as obtained by the above method.

### Anomalous Trajectory Analysis

While tracking provides a powerful tool to observe a vehicle in traffic, vehicle tracking and trajectory analysis (such as by using the FVC or other cameras) can be the part of the system that can detect anomalies in the approach. Some embodiments analyze the vehicle trajectory without human supervision, and can flag suspicious vehicles for further inspection. At least one embodiment of this process has two steps—mapping the vehicle's trajectory onto ground co-ordinates and detecting patterns of anomalous activities.

Processing the vehicle's trajectory in ground co-ordinates (for example, measuring distances in feet rather than pixels) can be beneficial because it allows operation in more operator-friendly units, and also removes the non-linear shape caused by the camera's perspective and road surface. This is evident in FIG. 3.3.16 where the trajectory corresponding to constant speed is highly non-linear. Thus, some embodiments use a transform that results in almost linear trajectories whenever a vehicle is being driven at a constant speed.

The above transformation can be achieved by first calibrating the camera—obtaining the true mapping between pixel positions and the ground positions, such as by driving a training vehicle at a known constant speed. The trajectory of this vehicle can be smoothened by applying a polynomial curve-fitting, and in some embodiments abstracting the transform into a look-up table (LUT). Thus, at the end of the calibration process, a look-up table which can be indexed with a pixel position and returns the ground position (distance from the camera or distance from a distant reference point) is obtained. This LUT can be used for subsequent vehicles to obtain the ground positions per frame, and the operation is not a limitation. Results of performing this co-ordinate mapping operation on the previously presented trajectories are shown in FIG. 3.3.17.

The transformed trajectories can then be analyzed to detect unusual behavior like unexplained slowing, stopping or sharp acceleration in some embodiments. This can be performed in two stages—learning the “normal” response of the vehicle when it is far from the camera, and detecting deviations from this normal behavior as the vehicle gets closer. We can characterize the response in terms of the approximate instantaneous speed, defined as:

g(t)=x(t)−x(t−1),

where x(t) represents the ground position of the vehicle at time instant t. The average speed ĝ can be computed when the vehicle is in the upper half of the field of view (FOV),

ĝ=(1/N)Σg(t),

where N is the total number of time instants when the vehicle is in the upper FOV.

Once the vehicle enters the lower half of the FOV, the instantaneous speed can be compared with ĝ for every time instant in some embodiments. An anomaly can be detected whenever the difference exceeds a threshold for a predefined number of consecutive frames (or, in alternate embodiments if g(t) becomes negative). FIG. 3.3.18 shows the decision markers generated by an example system when the above method is applied on a test vehicle executing different types of maneuvers—(a) near-uniform speed of 30 mph, (b) slow down followed by a sharp acceleration, and (c) U-turn. The circles represent time instants when the approach is adjudged “normal” by the system, while the crosses represent the suspected anomalies.

### Sensing and Analysis of Vehicle Dynamics and Vehicle Suspension Dynamics

Increased weight within a vehicle (e.g., a payload of explosives, such as those in excess of 100 kg) will generally alter how the vehicle responds dynamically to different roadway conditions (e.g., a speed bump, rumble strips). In some embodiments, measuring the dynamic response of the vehicle, in possible coordination with data from the video array that would indicate anticipated vehicle behavior, can allow for detection of suspicious vehicle conditions. Various vehicle components can be analyzed for their response to roadway conditions, such as the wheel, tire, axle, frame, body, engine, suspension component, drive component, or braking component.

The stiffness of the coil or leaf springs in the suspension system typically dominates the vehicle response. The suspension of the vehicle is generally tuned to meet the comfort needs of the passengers. The human body is prone to discomfort at certain frequencies. The sprung mass's (car without wheels) natural frequency is generally between 1 and 2 Hz for a ride that feels comfortable and natural for the passengers, and the unsprung mass's natural frequency is generally between 10-11 Hz. Pitching motion is more irritating to passengers than the bouncing motion, as such many suspensions are designed to translate pitching motion into vertical motion, such as by making the front suspension's natural frequency less than the rear's. With this tuning, a bump that is transmitted into the front suspension causes pitching motion, but when the rear wheels hit the bump, they move at a higher frequency than the front and quickly come into phase, translating pitching motion into vertical motion of the body. Severe passenger discomfort can occur when the vehicle vibrates between 4-8 Hz. Discomfort in this range is due, at least in part, to the abdominal cavity resonance. Vehicle vibrations below 1 Hz will cause sea sickness. Due to these and other issues, a typical vehicle suspension is tuned to have its body modes between 1-2 Hz and the wheel hop modes between 10-11 Hz. These modes are when the amplitude of vibration will be the greatest (i.e. the resonances). Embodiments of the present disclosure assume that these suspension characteristics apply to the vehicles being evaluated, which greatly reduces the complexity of examining vehicle dynamics across the thousands of types of cars on the roadway.

The suspension can be analyzed assuming it contains two elements: stiffness and damping. The coil or leaf springs provide the stiffness component, which absorbs the shock of road irregularities. The damping dissipates the energy transferred to the vehicle through the road irregularity. When a vehicle's suspension degrades, typically the damping component—i.e. the shock absorbers—typically are replaced. The damping typically has minimal effect on the body and wheel hop modes. When driving a vehicle with bad suspension, the passengers do not generally get sick from the modes shifting bellow 1 Hz or within the 4-8 Hz range. Examining the vehicle dynamics for potential VBIED detection has potential benefits since passenger vehicles have similar modes to ensure occupant comfort and bad shock absorbers generally have minimal impact on the vehicle modes. To determine methods for VBIED identification by vehicle dynamic response, the following tasks are used in some embodiments:
Vehicle modeling with a payload
Analysis on vehicle model
Simulation results from vehicle model
Vehicle model validation
Diagnostic cleat configuration and experimental results
Vehicle databases, parameter estimation, and sensitivity analysis

### Vehicle Modeling

In some embodiments the vehicle can be represented with a five degree-of-freedom (DoF) half car model, with parameter values that are derived from a combination of vehicle specifications and applied suspension design guidelines. A simulated VBIED can be added to the vehicle, and the changes in response can be monitored. The effects of perturbations and uncertainties in the vehicle parameters have been investigated, giving insight into the strengths of this method in addition to the challenges in applying this model to a large range of vehicles which do not have detailed specifications available. The 5 DoF model is in FIG. 3.4.1.

In FIG. 3.4.1, Mₛ is the sprung mass of the vehicle, Mᵣ is the mass of the rear unsprung mass (i.e. the mass of the rear wheel assembly and axle), M_{f} is the mass of the front unsprung mass, k_{f} is the suspension stiffness, k_{f} is the tire stiffness, b_{f} is the suspension damping, I_{cm} is the sprung mass moment of inertia, b is the distance between the center of gravity and rear wheels, a is the distance between the center of gravity and front wheels, c is the distance between the VBIED and center of gravity, and K_{L} is the equivalent stiffness of the simulated VBIED, b_{L} is the equivalent damping of the simulated VBIED. X₁ is the road input for the front wheels, and X₂ is the input for the rear wheels. The equivalent stiffness and damping for the load are properties of both the type of load and its placement. The model uses road displacement as input, and the force in the front and the rear tires as output. Various assumptions may be used in various embodiments, these include linear lumped parameter values and negligible tire damping. Tire damping may be assumed to be negligible because the damping is frequently much smaller than that of the shock absorbers.

The equivalent stiffness and damping for the load can be considered somewhat abstract properties.

The equivalent spring stiffness is dependent on both the mass's properties and placement. The equivalent spring is a series combination of stiffness between the chassis and the object. For example, this could mean the stiffness of the bushings where the cabin mounts to the chassis combined with the stiffness of the floor panel, the stiffness of the seat frame, the seat foam, and then the stiffness of the object itself. Since this is a series spring system, the equivalent stiffness is likely primarily dependent on the location of the load, because the compliant spring may dominate the equivalent stiffness, which would likely not be the stiffness of the material itself.

The equivalent damping is a measure of the vertical energy dissipated by the load per cycle of oscillation. Much like the stiffness, this could be affected by the placement of the load, but it can also be influenced by the type of load. For example, if objects were in a suitcase, moving front to back or side to side in response to a vertical excitation, they would be dissipating energy in the vertical direction. If the load was granular in nature, the friction in the particle motion would also dissipate energy in a different way than the previous example. Classifying these equivalent stiffness and damping parameters for different loads can be implemented in some embodiments for detecting the type of load in a vehicle.

Some embodiments analyze the change in vehicle response with the load modeled as an increase in the mass of the rigid body. Understanding the modes and mode shapes of the half car model and examining the changes in the system when the vehicle's values such as mass, center of gravity location, and moment of inertia are altered.

One example vehicle that was studied using these methods was a 2005 Chevrolet Astro van. A summary of vehicle parameters used in the ½ car model are in Table 3.4.2, and contain both known values and estimates based on those known values.

TABLE 3.4.2
Vehicle parameters used in ½ car model simulation

| | |
|---|---|
| Mₛ-Sprung Mass | 1737 Kg |
| M_{uf}-Front Unsprung Mass | 128 Kg |
| Mᵤᵣ-Rear Unsprung Mass | 165 Kg |
| I_{cm}-Moment of Inertia | 3741 Kg-m² |
| K_{f}-Front Spring Stiffness | 43089 N/m |
| Kᵣ-Rear Spring Stiffness | 52136 N/m |
| Kₜ-Tire Stiffness | 215000 N/m |
| b_{f}-Front Damping Constant | 2697 N-s/m |
| bᵣ-Rear Damping Constant | 2738 N-s/m |
| a-Distance front wheel to Center of Mass | 1.2979 m |
| WB-Wheelbase | 2.8207 m |

Table 3.4.3 contains the estimations of the body modes (bounce and pitch) and the wheel hop modes.

TABLE 3.4.3
Estimations of vehicle dynamic response

| Frequency | Interpretation |
|---|---|
| 1.0153 | Bounce |
| 1.2019 | Pitch |
| 8.52 | Front Wheel Hop |
| 9.60 | Rear Wheel Hop |

Initially a basic VBIED can be modeled as an increase in weight at the rear of the vehicle, which can be assumed to be part of the rigid body for modeling purposes in some embodiments. With this estimation, then, the mass increases the sprung weight, shifts the weight rearward, and increases the moment of inertia in pitch. Next, the stiffness and damping effects of the VBIED can be modeled.

The way that this change in the vehicle affects the modes of the model and the overall dynamics of the vehicle can then be examined. This includes a change in the transient response of the vehicle when excited by the road bump represented by the input functions. The first two modes shift downward with each incremental addition of weight in the trunk, and in the transient response, a large difference in the frequency of response is shown in the rear wheel force, largely independent of any shift in the front, see FIG. 3.4.4.

In FIG. 3.4.4, each line represents an addition of 100 kg of extra weight, and the top figure is the force in the front tires, the bottom the force in the rear. It is seen in these results that there is a measurable difference in the free response of the vehicle after the rear wheels traverse the speed bump. This measurable difference is measured by the peak-to-peak period of free response oscillation. As the weight increases, so does the period, which signifies the body mode frequency is decreasing. These results suggest that added weight in the vehicle can be identified by measuring the free response of the vehicle, identifying the vehicle parameters in Table 3.4.2 (total of 11), and using the ½ car model to estimate the expected free response and compare it to the actual response.

Next, the dynamic response of the Chevy van was examined (in some embodiments) when the simulated VBIED has two different load stiffness values (10,000 N/m, on the same order of magnitude as the suspension stiffness, and 100,000 N/m, on the same order of magnitude as the tire stiffness). Again, the dynamic response was examined by simulating the van driving over a speed bump. The difference in response to loads of 100, 200, and 300 kg placed 20 cm behind the rear axle are in FIG. 3.4.5 and FIG. 3.4.6, respectively.

When the payload stiffness is around the same magnitude of the vehicle suspension, there is both an increase in the peak-to-peak period and amplitude changes compared to the fixed VBIED simulation in FIG. 3.4.4. In FIG. 3.4.6 the stiffness between the vehicle and simulated VBIED is high; this is equivalent to the VBIED being part of the vehicle. If the VBIED has different dynamic characteristics than normal loads (i.e. different stiffness and damping) then the free response of the vehicle can be used to classify the type of material within the car.

It can be difficult to measure the free response using the traditional diagnostic cleats associated with some embodiments of the current disclosure. Alternate embodiments extend the cleat with embedded sensors to enhance its ability to measure this free response.

The ½ car model uses information on multiple (e.g., 12) parameters. Current vehicle databases may contain less than all of these parameters and some embodiments estimate the missing parameters, as discussed below.

The 5 DoF vehicle model can be used to examine how different types of loads affect the vehicle response (e.g., luggage, bird seed, liquid, etc.) Understanding the dynamic characteristics of normal and VBIED materials can help determine how to classify the load. To classify different load types, measurements on the stiffness and damping characteristics that exist between the car and within the material is performed.

Because the ½ car simulation contains many parameters, an analysis can be done to determine what parameter changes have the largest influence on the dynamic response. A Monte Carlo simulation may be carried out with the variables being randomized, and the resulting natural frequency may be examined against the parameter. In one example, a total of 100 random simulations were examined using the vehicle parameter ranges in Table 3.4.7.

TABLE 3.4.7
½ car model parameter ranges tested in Monte Carlo simulation

| Parameter | Min | Average | Max |
|---|---|---|---|
| Mtotal (kg) | 1905.043 | 1929.064 | 1965.671 |
| Ms (kg) | 1678.147 | 1734.022 | 1775.265 |
| Muf (kg) | 87.75861 | 118.6368 | 152.7687 |
| mur (kg) | 51.44008 | 76.40528 | 106.4748 |
| kf (N/m) | 37710.9 | 38622.52 | 40000.92 |
| kr (N/m) | 36448.99 | 37641.55 | 38874.2 |
| Kt (N/m) | 379865.8 | 390722.9 | 402581.1 |
| Bf (N-s/m) | 3837.436 | 4669.546 | 5322.307 |
| br N-s/m | 3688.16 | 4487.901 | 5115.269 |
| Icm (kg-m\hat{ }2) | 1982.211 | 3489.701 | 4135.933 |
| a (m) | 1.054434 | 1.078963 | 1.112025 |
| B (m) | 1.602975 | 1.636037 | 1.660566 |

The results of the Monte Carlo simulation showed that the moment of inertia in pitch was the dominant influence on the natural frequency of the vehicle, and could be identified despite changes in other parameters. FIG. 3.4.8 shows the plot of the first natural frequency versus four different parameters. This shows the strong dependence on the moment of inertia.

To validate these conclusions, a simple one-at-a-time sensitivity analysis can be done with each parameter varying from 90-110% of the original value, starting with the values as presented in Table 3.4.2 for the Chevrolet Astro. A linear regression was done on the plots of each parameter versus each natural frequency. Table 3.4.9 presents the results; showing the percent change in each natural frequency for a percent change in the parameter.

TABLE 3.4.9
Sensitivity Analysis results comparing % change in mode
versus % change in vehicle parameter

| | | | ωₙ 3-front | ωₙ 4-rear |
|---|---|---|---|---|
| | ωₙ 1-bounce | ωₙ 2 -pitch | wheel hop | wheel hop |
| K_{f} | 0.455433 | 0.008825 | 9.54E−07 | 0.0361388 |
| Kᵣ | 0.008848 | 0.447742 | 0.0439653 | −9.24E−07 |
| Kₜ | 0.025535 | 0.031042 | 0.4693006 | 0.4751667 |
| Mₛ | −0.21391 | −0.300304 | 0.0062603 | 0.0061571 |
| M_{uf} | −7.30E−05 | 4.23E−06 | −5.41E−06 | −0.501995 |
| Mᵤᵣ | 2.05E−06 | −0.000172 | −0.5018927 | −5.56E−06 |
| I_{cm} | −0.2932 | −0.21966 | 0.0066155 | 0.0048132 |
| b_{f} | 0.024443 | −0.002496 | 1.02E−07 | −0.021941 |
| bᵣ | −0.00214 | 0.028041 | −0.0259016 | 5.66E−06 |
| a | 0.446703 | −0.44868 | 0.0112991 | −0.009567 |

Large values in Table 3.4.9 indicate that the parameter has a greater influence on the specified mode. The first two modes are typically of more interest since they relate to the body mode (1-2 Hz) for the vehicle and will typically be the main cause of differences in the peak-to-peak measurement of the free response. To have an accurate representation of a vehicle's dynamic response, one or more of the suspension stiffness, sprung mass, moment of inertia, and location of the center of gravity may be evaluated. To make this vehicle dynamic analysis practical for use, values of these parameters for the vehicles within the United States can be utilized.

Some known databases capture vehicle characteristics of interest, see Table 3.4.10.

TABLE 3.4.10
Listing of vehicle databases

| Parameter | Availability | Database |
|---|---|---|
| Vehicle Mass | Widespread | CARSP[6] |
| Moment of Inertia in Pitch | Limited | NHTSA[7] |
| Weight Distribution | Widespread | CARSP[6] |
| Wheel Base | Widespread | CARSP[6] |
| Spring Stiffnesses | None | — |
| Damping Coefficients | None | — |
| Tire Stiffnesses | None | — |
| Unsprung masses | None | — |

Table 3.4.10 identifies that vehicle weight and dimensions are available in the CARSP database, which accounts for over 20,000 vehicle types driven in Canada. Though spring stiffness is not available in any databases, it can be estimated. Determining the sprung mass and unsprung mass given the total vehicle mass can be done in one embodiment by estimating the unsprung mass as a percentage of total vehicle. Depending on vehicle characteristics such as drive wheels and suspension choice, the total unsprung weight and the relative weights of the front and rear will typically vary. For the following equations, the rear is assumed to have more unsprung mass, as is the case with the van that will be tested. With this information and the natural frequency of the tire hop mode, tire stiffnesses can also be estimated. These estimation techniques can then produce of the model parameters for a vehicle given basic dimensions and weight, which are widely available for most makes and models. The stiffness in the front and rear suspension can be estimated by:

$${k}_{f} = \frac{{{k}_{T} ( {f}_{nf} * 2 π )}^{2} {m}_{f}}{{k}_{T} - ( {f}_{nf} 2 π ) {m}_{f}}$$

$${k}_{r} = \frac{{{k}_{T} ( 2 π \sqrt{1.21 * \frac{\% F}{\% R}} {f}_{nf} )}^{2} {m}_{r}}{{k}_{T} - {( 2 π \sqrt{1.21 * \frac{\% F}{\% R}} {f}_{nf} )}^{2} {m}_{r}} .$$

(note: % F and % R are the weight distributions front and rear, and mᵣ and m_{f} are % F*mₜₒₜₐₗ and % R*Mₜₒₜₐₗ) respectively, and f_{nf} is the selected frequency for the front wheel quarter car model)

kᵣ=(fₕₒₚ2π)²*M_{U}−k_{w}

(Where k_{w} is k_{f} or kᵣ, and M_{U} is M_{uf} or Mᵤᵣ) Rearranged from Eqn. (7).

m_{uf}=0.4*0.13*mₜₒₜₐₗ

mᵤᵣ=0.6*0.13*mₜₒₜₐₗ

b_{f}=2*ζ\sqrt{k_{f}*m_{f}}

bᵣ=2*ζ\sqrt{kᵣ*mᵣ}

l_{cm}=DIP*a*b*mₛ [3]

mₛ=0.87*mₜₒₜₐₗ

Expressions for the front and rear stiffness values are derived from equations in [3]. The damping ratio, ζ can be estimated as 0.3.

The half car model can be used to understand how an added load will affect the vehicle's dynamic response, and suggest methods for measuring this change. The model shows that excitation of the rear wheels of a vehicle typically produce larger pitch motions than excitation of the front wheels. The equations of motion take the following form, with k₂>k₁ and L₂>L₁.

$$\{ \begin{matrix}{X}_{f} \\ {Θ}_{f}\end{matrix} \} = \frac{1}{Δ} [ \begin{matrix}A & B \\ B & C\end{matrix} ] \{ \begin{matrix}{k}_{1} H \\ - {k}_{1} {L}_{1} H\end{matrix} \} and \{ \begin{matrix}{X}_{r} \\ {Θ}_{r}\end{matrix} \} = \frac{1}{Δ} [ \begin{matrix}A & B \\ B & C\end{matrix} ] \{ \begin{matrix}{k}_{2} H \\ {k}_{2} {L}_{2} H\end{matrix} \}$$

In addition, payloads are known to typically affect the pitch frequency more than the bounce frequency. An added payload generally decreases the pitch frequency towards the bounce frequency. The first two natural frequencies (for bounce and pitch motion) may be derived from the following equation.

$${ω}_{1 , 2}^{2} = \frac{1}{2} [ \begin{matrix}\frac{{k}_{1} + {k}_{2}}{m} + \frac{{k}_{1} {L}_{1}^{2} + {k}_{2} {L}_{2}^{2}}{{J}_{o}} ∓ \\ \sqrt{{( \frac{{k}_{1} + {k}_{2}}{m} + \frac{{k}_{1} {L}_{1}^{2} + {k}_{2} {L}_{2}^{2}}{{J}_{o}} )}^{2} - \frac{4 {k}_{1} {{k}_{2} ( {L}_{1} + {L}_{2} )}^{2}}{{mJ}_{o}}}\end{matrix} ]$$

An effective method for finding anomalies in dynamic systems is to create a feature vector that combines several factors that will change when an anomaly is introduced and is used in some embodiments. Taking the difference and the sum of the squares of the first two natural frequencies will isolate the first and second terms in the above equation. Plotting these feature vectors against one another, a trend can be found between standard and loaded vehicles, as seen in FIG. 3.4.11, with Series 1 representing various unloaded vehicles and Series 2 representing the same vehicles with a simulated 100 kg payload in the rear of the vehicle.

### Testing

An experiment was performed according to some embodiments on a 2005 Chevrolet Astro van to analyze the vehicle's dynamic response to a road bump excitation and compare that response to that of the van with 210 lbs of weight in the trunk to simulate a small VBIED.

PCB 3711D1FA20G DC accelerometers were mounted on the vehicle's chassis at the front and back of the vehicle, and PCB Y353B16 ICP accelerometers were placed on the front and rear unsprung masses as well, on the bottom of the rear axle housing for the rear unsprung mass, and at the bottom of the upper ball joint for the front. These accelerometers were then routed into the vehicle, where a data acquisition system collected data at 5000 Hz. In addition to vehicle mounted accelerometers, the road bump that excites the vehicle was also instrumented with tri-axial accelerometers. The vehicle mounted accelerometers were used to simplify analysis of the system. The rubber road bump was not thoroughly modeled at the time of the experiment and, in order to directly compare to simulated data, accelerometers were mounted on the vehicle.

Although vehicle-mounted accelerometers were used in embodiments that were tested, other embodiments utilize additional methods for sensing vehicle movement, and in certain embodiments vehicle axle movement. For example, some embodiments utilize remote methods, such as laser (and/or acoustic) vibrometers and/or velocimeters, to detect the motion of a portion of the vehicle, such as the axle.

Alternate embodiments utilize instrumented cleats and sensors used in conjunction with cleats as depicted in FIG. 3.4.09, and as disclosed in International Patent Application Nos. PCT/US09/057919, filed 22 Sep. 2009 (titled METHODS AND APPARATUS FOR DIAGNOSING FAULTS OF A VEHICLE, attny. docket no. 17933-90485), and PCT/US12/029954, filed 21 Mar. 2012 (titled EXTENDED SMART DIAGNOSTIC CLEAT, attny. docket no. 17933-96475), the entireties of which are hereby incorporated by reference in their entireties.

The vehicle was driven over the cleat at 5 mph ten times in each direction for each loading scenario: one time unloaded, one time with 210 lbs of sand bags in the trunk. Data was collected with both on-board sensors and those integrated into the cleat.

In one embodiment the first two modes were analyzed, although this analysis may be somewhat challenging. The data from the diagnostic cleat can include little frequency content below 10 Hz, and when the data from on-board accelerometers is transformed with the Fast Fourier Transform (FFT), it is possible for there to be no shift in the natural frequency observed for the first mode. The limited frequency content of the data from the cleat can be attributed to the tires being in contact with the cleat for a small period of time—much less than a period of 1 Hz response. The difficulty in distinguishing a change using a FFT on the data from on-board accelerometers may be due, at least in part, to the limited resolution of the FFT due to the sample duration time.

An analysis of the peak to peak time for the filtered angular acceleration signal according to some embodiments did reveal a change in the vehicles free response natural frequency. Comparison of the frequency of the free response before and after the rear wheels hit reveal a trend much like that seen in the transient response simulation—the front wheel response changed little, but the change became apparent after the rear wheel hits, as in FIG. 3.4.4. FIG. 3.4.11_A illustrates this. The red line (dashed line) is the loaded scenario, while the black (solid line) is the baseline.

Before the rear wheels hit, the responses between the loaded and unloaded case were nearly identical, but after the rear wheels hit, the loaded scenario responded at a lower frequency, quickly coming out of phase with the baseline signal. This example demonstrates the usefulness of comparing the free response frequency of the vehicle after the front wheels hit the road bump to that of the vehicle after the rear wheels hit in some embodiments of the present disclosure. This can be useful when analyzing a vehicle without extensive modeling of its own parameters because the signal is being compared to itself.

The data can be used in MATLAB for a parameter estimation study to validate the half car model, and determine the feasibility of directly estimating vehicle parameters from the measured response data. This estimation estimates the input function by integrating the front and rear wheel accelerations twice during the time of contact. The results of this estimation show that the model can describe the data well, but some parameter values are closer to estimates than are others. The parameter estimation estimates the mass close to the expected value, but other values such as the spring rates can turn out to be higher than expected. While these differences may be attributable to several causes, one of these is potentially the nonlinearity of the spring-damper suspension. The suspension is generally considered to be highly nonlinear, dampers, which have up to three times the damping in compression than in rebound.

### Statistical Analysis

Another indication of a potential VBIED within a vehicle evaluated in some embodiments is excess weight. Historically, VBIEDs can range from 100 lbs to over 1000 lbs. To conceal the excess loads, the terrorist can alter the vehicle's suspension to prevent the vehicle from sagging. Such a change in the suspension will cause the body mode of the vehicle to change, which can be detectable in the peak-to-peak frequency after the vehicle traverses a cleat. If the suspension is not altered, the body mode will change since the body mass is greater. This body mode change can be measured with the peak-to-peak frequency after the car traverses the cleat and then can be used to estimate the vehicle weight.

Using the CARSP database, a sensitivity analysis can identify the vehicle parameters that can identify if a car is outside its expected weight range. When performing this analysis, it can be assumed that the passenger weight is known. Due to this assumption, dependence on another sensor can be used to estimate the passenger weight (one reason for using embodiments with a multiple sensor approach). Also, the sensitivity analysis can identify what the other sensors should be able to measure to determine if the vehicle is outside its expected weight range. See FIG. 3.4.12.

FIG. 3.4.12 and similar figures can be used in some embodiments to identify the vehicle parameters needed to identify if a vehicle has excess weight. The vertical axis identifies the percentage of vehicle types excess weight can be identified on in the CARSP database. The horizontal axis identifies the minimum detectable excess weight if the passenger weight is known. The sensitivity identified of six parameters. For example, if only the overall length vehicle parameter is known, excess weight of 1000 kg may only be identified on approximately 20% of vehicle types. If the vehicle's overall length and width can be measured, excess weight of approximately 65% of vehicle types in the database may be identified. If the CARSP database contained the length and width dimensions of vehicles on the road, then the cleat technology may identify excess weights of 1000 kg in approximately 65% of the vehicle types on the road. A majority of VBIEDs do not exceed 1000 kg. If a common VBIED was 200 kg, approximately 5% of vehicles in the database may be identified with this excess load. Embodiments utilizing sensor technologies that can measure wheel base, measure height, identify make, and identify the model of the vehicle, approximately 85% of the vehicle types can be identified.

### Sensing and Analysis of Automobile Panel Vibrations

Cavities in automobiles, such as those behind body panels (e.g., panels that can conceal objects such as door panels, bumper covers, metal or plastic body sections, etc.), have been used to conceal explosive or illegal material. Non-contact laser vibrometry measurements of the outer skin used in some embodiments of the present disclosure can reveal material hidden within vehicle cavities. When combined with data from the make and model of the car, a deviation from baseline could indicate suspicious payloads behind the panel. The vibration of the door panel was studied to determine possible means to identify material hidden within the door.

A common vehicle door assembly has 4 main parts: the outer door skin, inner door frame, window module, and interior trim module. The window module includes the glass and a mechanism to lower the glass into the door. The interior trim module generally has an integrated armrest, storage compartment, and radio speaker.

The outer door skin is typically connected directly to the inner door frame and is not attached to the window module. The vibration of the outer door skin can be measured by the laser vibrometer. The inner door frame determines the boundary condition of door, which is used to determine the mode shape of the panel in some embodiments.

According to some embodiments, a door can be modeled with what is known to those skilled in the art as a “perfectly clamped” or “perfectly constrained” boundary condition on the sides and bottom of the door, constraining the rotational and translational motion. The top boundary condition can be difficult to model because of the elastic seal between the glass and door skin. The boundary condition connection of the door skin to the door glass can be modeled as a spring-damper system in some embodiments.

According to one embodiment, modeling and experiments were performed and used to validate the other. ABAQUS finite element method software can be used to model the door panel vibration. The first few natural frequencies of the modeled door panel vary with dimension, thickness, material, and curvature, but stay generally constant with geometry.

According to one embodiment, modal impact testing was performed on the front passenger side door panel of a stationary van. Accelerometers were located in the top, middle, and bottom part of the door skin, below the glass. The panel was impacted on two different mesh grid with varying dimensions (8×7 and 15×3 points across the door). The results were analyzed in MATLAB to produce the mode shape of door vibration at each natural frequency. The car door was tested as manufactured and with 8, 16, and 32 pounds attached to the door panel. The weights and their location on the door change the door panel's mode shapes and amplitudes.

The next experiment according to one embodiment simulated the behavior of a screening process using accelerometers attached to the door panel in lieu of a laser vibrometer. A laser vibrometer was not available for this testing; however, accelerometers attached to the door measured the same vibration signature as a vibrometer could. An unloaded door was tested along with various loading conditions.

### Panel Modeling

Finite element modeling can be performed in some embodiments to approximate the modal frequencies of a panel, e.g., a door panel. The model can include the outermost door skin due to the vibration of the outer panel is assumed to be independent of the inner parts (window module opening and interior trim module). The finite element model can be used to analyze the modal frequency changes with respect to dimension and mass of the panel representing different models of cars.

Sensitivity analysis is used in some embodiments to determine the variability of modal frequencies in the door panel. The first analysis can study how the first modal frequency varies with material selection. The second analysis can be conducted with respect to the door mass and corresponding change in dimension. The last analysis can be conducted with respect to the shape of the door panel.

Table 3.5.1 below compares the first five modal frequencies for sedan and van doors made of steel and aluminum.

TABLE 3.5.1
First modal frequency variation of car door panels with door material.

| | | Car Model | | | |
|---|---|---|---|---|---|
| | | GS 300 4 | GS 300 4 | Chev | Chev Astro |
| | | door Sedan | door Sedan | Astro Van | Van |
| Material | | Steel | Aluminum | Steel | Aluminum |
| Density | | 7700 | 2700 | 7700 | 2700 |
| Modulus of | | 210 | 69 | 210 | 69 |
| Elasticity | | | | | |
| Poisson's Ratio | | 0.31 | 0.35 | 0.31 | 0.35 |
| MODE | 1 | 48.5555 | 46.4504 | 30.0179 | 28.1187 |
| | 2 | 83.8551 | 80.2184 | 49.1125 | 46.005 |
| | 3 | 108.616 | 103.904 | 71.0264 | 66.532 |
| | 4 | 138.851 | 132.826 | 79.2478 | 74.2331 |
| | 5 | 149.746 | 143.248 | 93.2832 | 87.38 |

Modeling showed that the door's modal frequency can be insensitive to whether the door is made from aluminum or steel. This may be because the increase in stiffness for steel is counteracted by the increase in density; the frequency varies proportionally with stiffness and inversely with density, and would therefore remain constant.

Table 3.5.2 below lists the variation of the first and second modal frequencies of a sedan car door with the percent mass change of the original door shape.

TABLE 3.5.2
First and second modal frequency variation with
change in the percent mass.

| % | MASS | AREA | Mode | |
|---|---|---|---|---|
| MASS | (kg) | (m²) | 1 | 2 |
| 50% | 21.0 | 0.71 | 60.1 | 103.8 |
| 70% | 29.4 | 1.00 | 42.9 | 74.1 |
| 90% | 37.8 | 1.28 | 33.7 | 58.3 |
| 100% | 42.0 | 1.40 | 30.4 | 52.7 |
| 120% | 50.4 | 1.70 | 25.2 | 43.5 |
| 140% | 58.8 | 2.00 | 21.8 | 37.8 |
| 150% | 63.0 | 2.13 | 20.1 | 34.8 |

The mode 1 data points are depicted in FIG. 3.5.3.

This analysis shows that a linear change of mass results in a non-linear change in the modal frequency of the door panel. As the dimensions are increased, the change in frequency is plotted on the chart.

TABLE 3.5.4
First mode of Astro van and GS 300 door panel with linear mass change.

| No | % | Mass | Area | 1st Mode | 2nd Mode |
|---|---|---|---|---|---|
| Cases | mass | (kg) | (m\hat{ }2) | Astro Van | GS 300 |
| 1 | 50% | 21.0 | 0.71 | 59.71 | 60.15 |
| 2 | 70% | 29.4 | 0.99 | 42.86 | 42.94 |
| 3 | 90% | 37.8 | 1.28 | 33.44 | 33.76 |
| 4 | 100% | 42.0 | 1.42 | 30.17 | 30.50 |
| 5 | 120% | 50.4 | 1.70 | 25.15 | 25.21 |
| 6 | 140% | 58.8 | 1.99 | 21.65 | 21.88 |
| 7 | 150% | 63.0 | 2.13 | 20.00 | 20.15 |

The Pontiac GS 300 and Chevrolet Astro van door panels have different geometries and boundary conditions. In these cases, the sides have a “perfectly constrained” boundary condition. The first modal frequencies of the Astro van and GS 300 are similar, as seen in Table 3.5.4 above. Therefore, it can be concluded that shape changes have little effect on the modal frequency in this example.

### Experimental Results

According to some embodiments, several experiments were conducted to gauge the accuracy of the model and the viability of testing the vibration of car doors of rolling vehicles. Modal testing on the front door was used to verify the results found from the finite element model. Measurement of door vibration on moving vehicles was conducted to simulate the behavior of non-contact laser vibrometry. This experiment made use of accelerometers attached on the panel to attain real-time measurements. See FIG. 3.5.5.

In the cases indicated in Table 3.5.2, impact testing was performed on the grid indicated. The frequency response function between the input force of the hammer and the acceleration measured on the panel can be analyzed to give the mode shapes and natural frequencies of the door panel.

The vibration measurements on moving vehicles used an accelerometer on the door panel in lieu of measurement using a laser vibrometer. The excitation force on the door is from the motion of the car over a cleat on the road surface. The recorded vibration signature collected during the vehicle pass-over contains peaks that correspond to modal frequencies of the door. The pass-over measurement should produce results that complement the modal testing. The results from this experiment indicate that door vibration may be analyzed over a smooth roadway, as results obtained as the vehicle passed over the cleat were not always consistent with the experiments obtained on the smooth road.

The modeled resonant frequencies of the Chevrolet Astro van were similar to those found through modal impact testing. Testing found that the door glass contributes to the door skin's resonant frequencies. There were also some trends found during testing that might indicate material loaded onto the door.

FIG. 3.5.7 reflects the analysis mode shape and the experimental mode shape according to one embodiment.

TABLE 3.5.8
Frequencies of analysis and experimental of some mode numbers

| | Modeling Resonant | Modal Impact Testing |
|---|---|---|
| Mode Number | Frequency (Hz) | Resonant Frequency (Hz) |
| 1 | 56 | 59.6 |
| 2 | 72 | 68.3 |
| 3 | 81 | 72.3 |
| 4 | 89 | 82 |

Further refinement of the finite element model, particularly in regards to the boundary conditions of the door, can produce more accurate results in some embodiments. For example, a car door usually has seal strips, which add complicated spring-damper type boundary condition while some models in some embodiments use clamped boundary conditions.

Two trends were observed that may indicate additional mass upon the door panel. First, the amplitude is typically reduced with increasing mass. Second, the additional mass tends to shift the resonant frequency of the door panel.

Amplitude Integration is a technique to indicate loading upon the door panel in some embodiments. The amplitude of the frequency response functions obtained during modal testing decreased in certain frequency ranges as the panel was loaded. The sandbags used as additional mass provide damping that decreased the vibration energy in the frequency response function.

The frequency response function was integrated from 0 Hz to 200 Hz over 45 mesh points to indicate an overall trend of reduced frequency response in at least one embodiment. FIG. 3.5.9 shows the results of integrating over the mesh points.

The bars in FIG. 3.5.9 represent a particular channel and loading condition and shows integrated amplitude normalized to the unloaded case. The 32 lb loading condition shows a 50 percent decrease in integrated amplitude on the third channel. The second channel shows the decrease in integrated amplitude. At the 32 lb loading condition, the integrated amplitude is 20% of the unloaded value. This is thought to be because channel 2 is located close to a sandbag, and is therefore more highly loaded than other channels. The amplitude integration method is therefore revealing of loaded door panels when the measurement is close to the loading position.

A second method of integration in at least one alternate embodiment compares each of the 5 different loading conditions [unloaded, 8 lbs mass, 16 lbs mass, 24 lbs mass, 32 lbs mass] and 3 different channels [accelerometer channel 1, 2, and 3]. The amplitude is integrated from 0-200 Hz across varying numbers of points. FIG. 3.5.10 shows the results of this method of integration.

The reduction of the integrated amplitude with increased mass occurs in the channels and points. Channel 2 shows a reduction of integrated amplitude compared to channels 1 and 3.

This is thought to be because the accelerometer is located near a sandbag, and regardless of the number of points integrated, the amplitude measured through channel 2 will be reduced compared to other channels.

Both amplitude integration techniques are indicators of car door panels loaded with mass behind the door.

In addition to the amplitude reduction of the frequency response, the loading conditions increased the first resonant frequency of the door panel according to some embodiments. This result may seem counterintuitive; typically adding mass will reduce the vibration of system. However, adding more mass will increase the mass of a system and also constrain the motion of the door, particularly at the location of added mass. Thus, in some embodiments, the added mass can be modeled as increasing the effective stiffness of the door panel in addition to increasing mass. If the mass loading contributes more effective stiffness than mass, the resonant frequency will increase. The results of modal testing on the loaded door panels are depicted in FIG. 3.5.11.

Resonant frequency shifting takes place in the channels and is seen in channels 1 and 3. The resonant frequency in both channels is increased by approximately 6 Hz with increased loading condition on door panel.

### SUMMARY

Modeling of a car door panel is performed in some embodiments using finite element software when given approximate dimensions of the door. The modeling successfully validated the experimental mode shapes despite its simplicity.

The amplitude integration technique is used in some embodiments to detect additional loading on door panels. The large difference in integrated amplitude can reveal whether the door has additional mass hidden behind the outer skin.

### Tire Acoustical Mode Analysis

Aspects of some embodiments detect anomalous conditions of automobile tires.

Anomalous conditions of the tires could be due to: (i) over-inflation of the tire to compensate for overloading of the vehicle, or, (ii) the insertion of foreign material into the tire air cavity. The use of acoustical measurements, in combination with data obtained from the other technologies, can be used in some embodiments to detect information about both tire pressure and interior fill material.

Between the rim and the carcass of the tire there is a volume that is filled with pressurized air to provide cushioning and support for the vehicle. Increased loading or decreased air pressure will cause deformation of the tire carcass. Large deformations would be clearly visible in a tire; in order to disguise this, the air pressure inside the tire can be increased to mask the effects of an exceptionally high load on the tire due to over-loading of the vehicle. Because higher air pressure in tires can be dangerous and make the tire more susceptible to blowouts, air pressures above the manufacturer's recommendation would be rare for a vehicle in normal use. Detection of anomalously high air pressure in a tire may be used in some embodiments as an indicator of suspicious behavior.

Recent events have shown that the smuggling of drugs in tire air cavities occurs at the border crossing between the United States and Mexico. While the exact method of hiding drugs in tires is unknown, inserting any foreign material into a tire cavity reduces the air space within the tire and increases the damping in the space. This reduction in air space would cause a change in the tire acoustical mode that would not be detectable by visual inspection of the tire, but which could be detected acoustically in some embodiments.

The tire acoustical mode is a property of the volume of air inside the tire and of the tire geometry. The frequency of the mode will vary with loading on the tire and tire rotation speed; for tires, this mode is generally located between 200 and 250 Hertz (Hz). The physical properties of the acoustical mode can allow for detection of anomalously high pressure or the insertion of foreign material into the tire cavity.

The tire acoustical mode transmits a force typically through the axle to the vehicle interior. The spectrum of the force transmitted to the axle shows a spike at the frequency of the acoustical mode. This axle force can cause audible tones inside the vehicle cabin at the frequency of the tire acoustical mode; therefore, control of the force exerted by this mode upon the axle is of concern in automobile design.

According to some embodiments of the present disclosure, a phenomenological model of the tire acoustical mode was developed. This model accounts for changes in the axle loading due to tire load and the separation of the mode into peaks (e.g., two peaks) due to both the effects of tire deformation due to loading and tire rotation speed. Tests were performed to verify the character of this model, which indicated the presence of an oscillatory force on the axle that corresponded with their predictions.

A model of the coupling between the tire acoustical mode and the tire structural modes was developed according to some embodiments. The structural modes of the tire correspond to vibration in the radial direction as well as along the sidewalls and the thickness (normal into the axis of rotation) of the tire. The acoustical mode in the tire can apply pressure normal to the tire surface, while transverse waves may propagate through the tire. The developed models for the effects of tire loading are due to tire deformation, accounting for the pressure inside the tire. It also presented methods for control of the acoustical mode, including the use of a resonating chamber or localized interior filling, thus demonstrating that the acoustical mode is modified or eliminated when the tire is partially or completely filled with a foreign material.

Another model used in some embodiments explored the vibration of the tire carcass and the resulting sound radiation. It developed FEM models for the tire that can take into account the sidewall thickness, tread compound, and tread variation around the tire. Their analysis yielded sound radiation from the components of the tire structure, and found that the tire acoustical mode was causing carcass vibration at the tire acoustical mode frequency.

Another model used in some embodiments analyzed tire vibration using a wavenumber decomposition technique. By plotting vibration data on wavenumber and frequency axes, this method allows for detection of various wave types in the tire, including the acoustical mode. Cut-on frequencies and wavespeeds can be determined by analyzing the data obtained from wavenumber decompositions.

The tire acoustical mode is generally caused by propagating waves in the fluid medium enclosed by the tire carcass and the rim. The circular shape of the tire uses acoustical modes within the tire cavity with an integer number of wavelengths along the tire circumference. The lowest mode of the tire interior volume will typically have a frequency equal to f=c/(πd), where c is the speed of sound in air (343 m/s at room temperature) and d is the mean tire diameter. For tires, the first acoustical mode generally occurs between 200-250 Hz, and higher modes may occur at nearly integer multiples of this first mode.

The mean tire diameter is calculated as the distance between the area centers of the tire structure on opposite sides of the tire. This area center is the transverse center of the air space within the tire. The location of the center is affected by the tire size and the rim size. The mean diameter of the tire can be approximated by the size of the tire: for example, a 235/70R15 tire has a height of 70 percent of its width of 235 mm that mounts to a 15 inch rim. This would lead to a mean tire diameter of 0.54 meters. The rim area is particular to the rim construction, but is less than the tire area, and will typically shift the location of the area center slightly. Both tire diameter and rim diameter can be estimated in some embodiments by visual inspection as well.

The lowest acoustical mode generally features two nodes and two antinodes along the tire circumference. Without loading, the tire structure is circularly symmetric, and the location of these nodes would be arbitrary with respect to the circumference. Under loading, however, the deformation of the tire causes the single tire mode to split into two tire modes, oriented in relation to the location of loading and having distinct natural frequencies. The lower frequency mode will have a node at the point of contact, while the higher frequency mode will have an antinode at the point of contact. FIG. 3.6.1 shows the splitting of the two modes when under deformation. The amount of separation between these two modes depends on the amount of deformation in the tire; this deformation is a function of the tire pressure, rim stiffness, and force applied to the tire.

Additional separation of the tire acoustical modes will typically occur when the tire rotates. The tire modes that are already split under loading will shift further apart with increased tire rotation speed. This rotation speed can be calculated from the tire radius and vehicle speed.

The tire acoustical mode generally has a sharp peak in axle force at the tire acoustical mode frequency. The sharpness of this peak is due to the small damping provided by air in the tire. Other tire structural effects occur at frequencies above 300 Hz; therefore, the peak associated with the acoustical mode can be detectable in the axle force. It is expected that this force can also cause pressure to be applied to the tire sidewall that will lead to radiation of sound to the exterior at the tire acoustical mode frequency.

The speed of sound of air is generally invariant with pressure; thus, increased pressure in the tire cavity does not affect the frequency of the tire acoustical mode (other than minute changes related to the change in tire diameter due to increased pressure in the tire). Changes in tire temperature can affect the speed of sound, but this change is generally small (approximately 2 Hz per 10 degrees Celsius). Tire size can be determined in some embodiments using the video technology discussed elsewhere in this disclosure, and that information can provide an estimate of the tire acoustical mode frequency that can provide a starting point for searching for the peaks caused by the tire acoustical mode. The static force on the tire due to vehicle loading can be estimated in some embodiments through the vibration measurement methodology also described in this disclosure, and the vehicle speed can be measured by radar or video means to obtain an estimate of the tire rotation frequency. A suspicious vehicle could be flagged if the data obtained by the measurements indicates that the tire size, loading, and rotation speed are not consistent with the expected acoustical mode frequencies, or if the acoustical mode appears to be absent.

### Testing

The tire acoustical mode for many tires has been well-documented with regard to the force it exerts on the wheel axle; however, little if any experimental work has been performed on its radiation as sound from the tire. According to some embodiments, several tests were performed to verify the findings of previous research and gauge their applicability to detecting the tire acoustical mode. These tests included optional vibration and acoustical measurements of the tire structure under point structural excitation, as well as optional acoustical measurements of radiated noise in response to drop tests of the tire. Measurements of vibration along the tire tread, vibration on the tire axle, and force input into the tire were optionally performed as well.

In one embodiment, the tires tested were Kelly Safari Signature 235/70R15 tires. These tires had defects; one tire had a gash along the sidewall rubber and another had areas of exposed tire belt along the tread. However, each of these tires with different defects yielded similar test results, and it is not thought that these defects affected any of the measurements pertaining to the acoustical mode in the airspace of the tire. The air space in this tire would typically indicate a tire acoustical mode at 200 Hz without taking into account the size of the tire rim. The rim geometry was not measured precisely; however, it was estimated to be approximately 3-5 cm deep, which would cause an increase of 10-15 Hz of the frequency of the tire acoustical mode. Thus the tire acoustical mode was expected to appear between 210 and 215 Hz.

One of the tires was filled with air, as would be a regular car tire. The second tire was filled with sound-absorbing material typically used as insulation for aircraft cabins. The insertion of the insulation material will generally reduce propagation of the acoustical mode in the tire and should reduce or eliminate any characteristics caused by the acoustical mode. The tire filled with insulation material was still inflated to provide stiffness in the tire equal to the air-filled tire. A photo of the tire being filled with insulation material is provided in FIG. 3.6.2.

According to at least one embodiment, vibration tests on the tire sidewall were performed to allow for wavenumber decomposition of the tire vibration signature. Use of a scanning laser vibrometer allows for automated measurement around the tire circumference. The individual measurement point around the tire sidewall produces a frequency signature of the vibration. The spatial variation of vibration with position on the tire at a single frequency can then be Fourier transformed with respect to position to generate a wavenumber decomposition of tire vibration. The wavenumber is the rate of change of phase with position, and for propagating waves should vary with frequency; the rate of that variation is inversely proportional to wave speed. Thus, a plot of the tire vibration as a function of the wavenumber and frequency can reveal lines that indicate the existence of waves propagating through the tire sidewall and their speed. FIG. 3.6.3 shows an example of transforming the tire vibration from position-frequency to wavenumber-frequency.

The tests were performed with tire pressures between 55 pounds per square inch (psi) and 30 psi in increments of 5 psi. Eighty (80) points along the tire sidewall were measured using the scanning laser vibrometer. The tire was mounted to an axle on a rigid stand and was excited using a small shaker attached to the tire treads through a point stinger. White noise was used to excite the tire, allowing for equal-force input at many frequencies. The acceleration at a point near the excitation, along with the input force voltage, was measured to provide normalization of the tire vibration. Measurements were performed at a 2000 Hz sampling rate using the laser vibrometer data acquisition system.

The results of the tire vibration measurements at the lowest and highest tire pressures tested are shown in FIG. 3.6.4 and FIG. 3.6.5, respectively. These results exhibited similar characteristics, with a low-speed wave having velocity between 32 and 48 meters per second cutting on around 50 Hz and a second wave cutting on around 280 Hz that increases in wavespeed until it matches the speed of the earlier cut-on wave. Neither of these wavespeeds was near the speed of sound, which is indicative of their traveling through the tire carcass structure. The wavespeed in the tire increased by 50% as the tire pressure changes from 30 to 55 psi (i.e., the slope of the lines in the wavenumber-frequency plots became steeper), indicating that the increased pressure likely causes stiffening of the tire carcass structure. The acoustical waves may be of relatively low amplitude in the tire sidewall vibration owing to the point excitation used in the test. The application of a uniform force over the contact patch, which more closely approximates the force applied to a loaded, rolling tire, may also be evaluated.

By examination of the results, evidence of the acoustical mode can be detected. Detailed sections of the wavenumber-frequency plots are shown in FIG. 3.6.6 and FIG. 3.6.7. The detail shows the area of the plot close to the expected tire acoustical mode frequency of 212 Hz and wavenumber of 2/d (approximately 4 m⁻¹ for the tested tire). The presence of a peak there is indicative of the tire acoustical mode. The peak is higher (in relation to neighboring characteristics) at 55 psi than at 30 psi; this is likely due to the increased loading the acoustical mode exerts on the tire structure at higher pressures. Note also that the features at higher wavenumbers (between 20 and 25 m⁻¹) that result from carcass vibration moved with inflation pressure, as expected, while the features related to the tire acoustical mode do not. These results thus demonstrate that the tire acoustical mode creates measurable vibration on the surface of the tire which presumably then causes sound to radiate from the tire.

In some embodiments, acoustical measurements were performed on both the air-filled and insulation-filled tires to detect a tone that could be measured exterior to the tire. A multi-microphone (e.g., four-microphone) acoustical array was set up adjacent to the tire, with additional optional measurements of the force input into the tire, the acceleration on the sidewall near the force input, noise from the shaker setup, and acceleration of the axle to which the tire is mounted. Four microphones may not have provided enough data for holographic visualization or wavenumber transforms of useful resolution, but provided evidence of the nature of the sound radiating from the tire structure. Both the air-filled and insulation-filled tires were tested from 60 to 20 psi in 5 psi increments. The data was sampled at 5120 KHz using a custom VXI-based setup. A picture of the setup is in FIG. 3.6.8.

Data from an accelerometer located on the axle was optionally used to provide a link between generally accepted knowledge on the tire acoustical mode and the collected data. The vibration of the axle at several representative tire pressures is shown in FIGS. 3.6.9 through 3.6.11. The air-filled and insulation-filled tires exhibit differences at 145 Hz and 211 Hz, with the air-filled tire displaying a lightly damped spike in axle vibration. It is not known what is causing the difference at 145 Hz, but the 211 Hz spike can generally be attributed to the tire acoustical mode. At lower pressures, both spikes disappear, which could indicate that the tire acoustical mode is exerting less force on the axle. From these results, filling the tire with a foreign material causes the tire acoustical mode to be suppressed. Thus, the absence of this feature in some embodiments indicates that the tire has a material in it other than air.

A comparison of the acoustical signature of the average radiation recorded by the four microphones is shown below in FIG. 3.6.12. At 212 Hz, there is a difference in the two tires' acoustical radiation signature, with the air-filled tire exhibiting a lightly-damped spike. Otherwise, there is little difference in the two tire acoustical signatures. This appears to indicate that the acoustical mode can be detected with microphones. As shown in FIG. 3.6.13, the spike at 212 Hz is much lower at 40 psi, and at 20 psi the acoustical radiation of the two tires is almost identical.

The radiation measured at the four individual microphones for an air-filled tire is shown in FIG. 3.6.14 and the corresponding results for the insulation-filled radiation tire are shown in FIG. 3.6.15. At 212 Hz, the sound pressure level radiated by the air-filled tire varies with position. This would indicate that the acoustical signature around the tire varies with position, and that spatial filtering may be used to enhance detection of the mode. Note that the radiation associated with the acoustical mode is absent from the corresponding insulation-filled results (FIG. 3.6.15).

During acoustical testing, it was noticed that the two tires exhibited different tonal characteristics when dropped on the floor. Subjectively, those differences were more different than those heard during the steady-state, point-force driven acoustical measurements. Drop tests of the tire were recorded to quantify these differences. The tire was dropped from a height of six inches and caught after a single bounce. Two microphones located approximately one foot from the tire were used to record the drop at a 5120 Hz sampling rate. The time histories of the drops were cropped to an equal length for the tests (regardless of the damping of the wave) to provide similar amplitudes for the test.

Results from the drop tests are shown below in FIG. 3.6.16 through FIG. 3.6.18. The spike at 212 Hz was considerably more prominent during the drop test than in the steady-state, point-force driven acoustical measurements. Further, the spike at 212 Hz is absent when the tire was filled with insulation material. One noticeable difference between the two types of tests was in the nature of the force distribution applied to the tire. Since force is applied to the contact patch in the drop test, as the tire would experience in normal operation, the results of the drop test were judged to be more representative of operational results. As expected, at lower pressures the acoustical signature from the insulation-filled tire begins to look identical to that of the air-filled tire.

As noted, the difference in prominence of the tire acoustical mode in the two different types of tests is thought to be due to the method of excitation. While the vibration excitation used in the steady-state tests was over a small surface, the drop test impacts the contact patch of the tire. The latter excitation will possibly cancel out higher-order vibrational modes propagating in the tire carcass and so will emphasize the lower order acoustical modes.

### SUMMARY

Acoustical measurements of the tire during vehicle pass-by can be used in some embodiments to detect the presence of anomalous tire conditions. Inconsistencies in the properties of the acoustical mode that are dependent on tire geometry, pressure, and loading indicate tampering with the tire from its standard state.

The tire acoustical mode is detected externally in at least some embodiments. This peak matches the frequency on the acoustical mode forces on the tire axle. This distinct peak at the frequency of acoustical mode resonance disappears when the tire is filled with a foreign material.

The data on tire size (obtained from the video analysis) and vehicle weight (obtained from dynamic analysis) can be used in some embodiments to indicate the position of an expected tire acoustical resonance.

Still further embodiments detect abnormally high pressure in the tire. While the existence of split peaks due to loading are evident in loading on the tire axle, twin peaks have not yet been seen in the acoustical signature of sound radiated from the tire. Furthermore, the deformation of the tire that causes split peaks is a function of both the loading on the tire and internal pressure. The sensitivity of the resonance peaks to tire pressure and loading may be used to estimate tire inflation pressure.

In addition to tire sound radiation, vehicle noise is also detected by the sensing modality in some embodiments. The use of beam forming arrays can be used to spatially filter the noise, amplifying the tire sound by utilizing the expected mode shape.

One or more of the various apparatuses, methods and systems for sensing and analyzing various vehicle characteristics discussed above can communicate with one another in alternate embodiments. As an example, FIG. 3.7.1 illustrates various participants in a system 100, all connected via a network 150 of computing devices. Some participants, e.g., subsystem 120, may also be connected to a server 110, which may be of the form of a web server or other server as would be understood by one of ordinary skill in the art. In addition to a connection to network 150, subsystems 130 and 140 may each have data connections, either intermittent or permanent, to server 110. In many embodiments, each computer will communicate through network 150 with at least server 110. Server 110 may also have data connections to additional subsystems as will be understood by one of ordinary skill in the art.

Each of subsystems 120, 130 and 140 may include one or more of the apparatuses and/or methods for sensing and analyzing a vehicle disclosed herein, such as a subsystem that analyzes the acoustical response of a tire as the tire rolls over a surface, analyzes one or more characteristics of a vehicle by analyzing at least one image of the vehicle, analyzes the response of a vehicle component to the vehicle moving over a surface, analyzes the response of a vehicle component to vehicle-generated vibrations, and/or measures the weight of the vehicle.

The computers used as servers, clients, resources, interface components, and the like for embodiments described herein can generally take the form shown in FIG. 3.7.2. Computer 200, as this example will generically be referred to, includes processor 210 in communication with memory 220, output interface 230, input interface 240, and network interface 250. Power, ground, clock, and other signals and circuitry are omitted for clarity, but will be understood and easily implemented by those skilled in the art.

With continuing reference to FIG. 3.7.2, network interface 250 in this embodiment connects computer 200 to a data network (such as a direct or indirect connection to server 110 and/or network 150) for communication of data between computer 200 and other devices attached to the network. Input interface 240 manages communication between processor 210 and one or more input devices 270, for example, microphones, pushbuttons, UARTs, IR and/or RF receivers or transceivers, decoders, or other devices, as well as traditional keyboard and mouse devices. Output interface 230 provides a video signal to display 260, and may provide signals to one or more additional output devices such as LEDs, LCDs, or audio output devices, or a combination of these and other output devices and techniques as will occur to those skilled in the art.

Processor 210 in some embodiments is a microcontroller or general purpose microprocessor that reads its program from memory 220. Processor 210 may be comprised of one or more components configured as a single unit. Alternatively, when of a multi-component form, processor 210 may have one or more components located remotely relative to the others. One or more components of processor 210 may be of the electronic variety including digital circuitry, analog circuitry, or both. In one embodiment, processor 210 is of a conventional, integrated circuit microprocessor arrangement, such as one or more CORE i7 HEXA processors from INTEL Corporation of 2200 Mission College Boulevard, Santa Clara, Calif. 95052, USA, or ATHLON or PHENOM processors from Advanced Micro Devices, One AMD Place, Sunnyvale, Calif. 94088, USA, or POWER8 processors from IBM Corporation, 1 New Orchard Road, Armonk, N.Y. 10504, USA. In alternative embodiments, one or more application-specific integrated circuits (ASICs), reduced instruction-set computing (RISC) processors, general-purpose microprocessors, programmable logic arrays, or other devices may be used alone or in combination as will occur to those skilled in the art.

Likewise, memory 220 in various embodiments includes one or more types such as solid-state electronic memory, magnetic memory, or optical memory, just to name a few. By way of non-limiting example, memory 220 can include solid-state electronic Random Access Memory (RAM), Sequentially Accessible Memory (SAM) (such as the First-In, First-Out (FIFO) variety or the Last-In First-Out (LIFO) variety), Programmable Read-Only Memory (PROM), Electrically Programmable Read-Only Memory (EPROM), or Electrically Erasable Programmable Read-Only Memory (EEPROM); an optical disc memory (such as a recordable, rewritable, or read-only DVD or CD-ROM); a magnetically encoded hard drive, floppy disk, tape, or cartridge medium; or a plurality and/or combination of these memory types. Also, memory 220 is volatile, nonvolatile, or a hybrid combination of volatile and nonvolatile varieties. Memory 220 in various embodiments is encoded with programming instructions executable by processor 210 to perform the automated methods disclosed herein.

Various aspects of different embodiments of the present disclosure are expressed in paragraphs X1, X2, and X3, as follows:

X1. One embodiment of the present disclosure includes a system, comprising: a system that utilizes information from two or more subsystems and detects irregularities in a vehicle, wherein said two or more subsystems are selected from the group consisting of subsystems A, B, C, and D; wherein subsystem A analyzes the acoustical response of a tire as the tire rolls over a surface; wherein subsystem B analyzes one or more characteristics of a vehicle by analyzing at least one image of the vehicle; wherein subsystem C analyzes the response of a vehicle component to the vehicle moving over a surface; and wherein subsystem D analyzes the response of a vehicle component to vehicle-generated vibrations.

X2. Another embodiment of the present disclosure includes a system that detects irregularities in a vehicle with at least one tire by analyzing the acoustical response of the at least one tire as the at least one tire rolls over a surface.

X3. Another embodiment of the present disclosure includes a system that detects irregularities in a vehicle by analyzing video images of vehicle.

X4. Another embodiment of the present disclosure includes a system that detects irregularities in a vehicle by analyzing the kinematic response of a vehicle component to external excitation.

X5. Another embodiment of the present disclosure includes a system, comprising: a receiver for receiving acoustical information of a vehicle tire as the vehicle tire rolls over a surface; and a processor that analyzes information related to the acoustical response of the vehicle tire as the vehicle tire rolls over the surface received by the receiver and detects irregularities in the vehicle.

X6. Another embodiment of the present disclosure includes a system, comprising: a camera for capturing video images of a vehicle; and a processor that analyzes the video images of a vehicle and detects irregularities in the vehicle.

X7. Another embodiment of the present disclosure includes a system, comprising: a sensor that receives information about a the kinematic response of a vehicle component to external excitation; and a processor that analyzes the information and detects irregularities in the vehicle.

X8. Another embodiment of the present disclosure includes a method, comprising: detecting irregularities in a vehicle with at least one tire by analyzing the acoustical response of the at least one tire as the at least one tire rolls over a surface.

X9. Another embodiment of the present disclosure includes detecting irregularities in a vehicle by analyzing video images of vehicle.

X10. Another embodiment of the present disclosure includes detecting irregularities in a vehicle by analyzing the kinematic response of a vehicle component to external excitation.

Yet other embodiments include the features described in any of the previous statements X1, X2, X3, X4, X5, X6, X7, X8 or X9, as combined with one or more of the following aspects:

Wherein at least one of the at least two subsystems receives data from the other of the at least two subsystems.

Wherein at least one subsystem detects the presence, absence or alteration of a particular acoustical mode of the tire as the tire rolls over the surface.

Wherein at least one subsystem detects the presence, absence or alteration of the 210 Hz acoustical mode of the tire as the tire rolls over the surface.

Wherein at least one subsystem compares a characteristic of the structural frequencies of the tire to the same characteristic of the structural frequencies of a properly inflated tire.

Wherein at least one subsystem compares a characteristic of the structural frequencies of the tire to the same characteristic of the structural frequencies of a properly inflated tire.

Wherein at least one subsystem evaluates differences between the structural frequencies of the tire and the structural frequencies of a properly inflated tire.

Wherein at least one subsystem evaluates differences in the Quality Factor between the structural frequencies of the tire and the structural frequencies of a properly inflated tire.

Wherein at least one subsystem indicates the presence of an irregularity in the vehicle when the difference in the Quality Factor between the structural frequencies of the tire and the structural frequencies of a properly inflated tire differ by more than 10%.

Wherein at least one subsystem indicates the presence of an irregularity in the vehicle when the difference in the Quality Factor between the structural frequencies of the tire and the structural frequencies of a properly inflated tire differ by more than 25%.

Wherein at least one subsystem indicates the presence of an irregularity in the vehicle when the difference in the Quality Factor between the structural frequencies of the tire and the structural frequencies of a properly inflated tire differ by more than 50%.

Wherein at least one subsystem includes an apparatus for inducing an acoustical response in the tire.

Wherein at least one subsystem includes a grooved road surface for inducing an acoustical response in the tire.

Wherein at least one subsystem includes a cleat for inducing an acoustical response in the tire.

Wherein the characteristic analyzed by at least one subsystem is the orientation of the body of the vehicle.

Wherein the characteristic analyzed by at least one subsystem is the front end up (or down) orientation of the body of the vehicle.

Wherein the characteristic analyzed by at least one subsystem is the shape of at least one of the vehicle's tires.

Wherein at least one subsystem analyzes one or more characteristics of the vehicle by analyzing a plurality of video images of the vehicle.

Wherein at least one subsystem analyzes the change in orientation of the vehicle body by analyzing a plurality of video images of the vehicle.

Wherein at least one subsystem analyzes the type, make, or model of the vehicle.

Wherein at least one subsystem includes a cleat for the tire to roll over and a sensor to measure the vibrations in the tire due to the tire rolling over the cleat.

Wherein at least one subsystem includes a laser vibrometer.

Wherein at least one subsystem analyzes the vibration of the external surface of a vehicle caused by operation of the vehicle's engine.

Wherein the system detects the presence of substances placed in vehicle cavities.

Wherein the irregularity detected by the system is the presence of material not part of the originally manufactured vehicle hidden within the vehicle.

Wherein the system detects abnormal tire pressure without physically contacting the tire.

Wherein the system analyzes whether the vehicle is oriented in a front end up or front end down orientation when compared with the a normally loaded vehicle.

Wherein the system analyzes the shape of at least one of the vehicle's tires.

Wherein the system detects irregularities in a vehicle by analyzing the kinematic response of the vehicle moving over a surface.

Wherein the system includes a textured surface over which the vehicle moves as the vehicle's kinematic response to the textured surface is analyzed.

Wherein the system includes a bump over which at least one vehicle wheel travels as the vehicle's kinematic response to the textured surface is analyzed.

Wherein the surface over which the vehicle wheel travels includes the sensor.

Reference systems that may be used herein may refer generally to various directions (e.g., upper, lower, forward, rearward, front, side, etc.), which are merely offered to assist the skilled reader in understanding the various embodiments of the disclosure and are not to be interpreted as limiting. Other reference systems consistent with that which is being described may be used to describe various embodiments as would be understood by one of ordinary skill in the art.

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Features of one embodiment may be used in combination with features of other embodiments as would be understood by one of ordinary skill in the art, whether or not explicitly described as such. One or more exemplary embodiments have been shown and described, and all changes and modifications that come within the spirit of the disclosure are desired to be protected.

What is claimed is:

1. A system, comprising:
a system that utilizes information from two or more subsystems and detects irregularities in a vehicle, wherein said two or more subsystems are selected from the group consisting of subsystems A, B, C, and D;
wherein subsystem A analyzes the acoustical response of a tire as the tire rolls over a surface;
wherein subsystem B analyzes one or more characteristics of a vehicle by analyzing at least one image of the vehicle;
wherein subsystem C analyzes the response of a vehicle component to the vehicle moving over a surface; and
wherein subsystem D analyzes the response of a vehicle component to vehicle-generated vibrations.

2. The system of claim 1, comprising:
subsystem E, wherein subsystem E includes analyzing the weight of the vehicle.

3. The system of claim 1, wherein at least one of the at least two subsystems receives data from the other of the at least two subsystems.

4. The system of claim 1, wherein subsystem A detects the presence, absence or alteration of a particular acoustical mode of the tire as the tire rolls over the surface.

5. The system of claim 1, wherein subsystem A compares a characteristic of the structural frequencies of the tire to the same characteristic of the structural frequencies of a properly inflated tire.

6. The system of claim 1, wherein subsystem A compares a characteristic structural frequency of the tire appearing between approximately 210-215 Hz to the same characteristic structural frequency of a properly inflated tire.

7. The system of claim 1, wherein subsystem A evaluates differences between the structural frequencies of the tire and the structural frequencies of a properly inflated tire.

8. The system of claim 1, wherein subsystem A evaluates differences in the Quality Factor between the structural frequencies of the tire and the structural frequencies of a properly inflated tire.

9. The system of claim 1, wherein subsystem A indicates the presence of an irregularity in the vehicle when the difference in the Quality Factor between the structural frequencies of the tire and the structural frequencies of a properly inflated tire differ by more than 10%.

10. The system of claim 1, wherein subsystem A includes an apparatus for inducing an acoustical response in the tire.

11. The system of claim 1, wherein the characteristic analyzed by subsystem B is the orientation of the body of the vehicle.

12. The system of claim 1, wherein the characteristic analyzed by subsystem B is the shape of at least one of the vehicle's tires.

13. The system of claim 1, wherein subsystem B analyzes one or more characteristics of the vehicle by analyzing a plurality of video images of the vehicle.

14. The system of claim 1, wherein subsystem B analyzes the type, make, or model of the vehicle.

15. The system of claim 1, wherein subsystem C includes a cleat for the tire to roll over and a sensor to measure the vibrations in the tire due to the tire rolling over the cleat.

16. The system of claim 1, wherein subsystem C includes a laser vibrometer.

17. The system of claim 1, wherein subsystem D analyzes the vibration of the external surface of a vehicle caused by operation of the vehicle's engine.

18. The system of claim 1, wherein the system detects the presence of substances placed in vehicle cavities.

19. The system of claim 1, wherein the irregularity detected by the system is the presence of material not part of the originally manufactured vehicle hidden within the vehicle.

20. A system, comprising:
a receiver for receiving acoustical information of a vehicle tire as the vehicle tire rolls over a surface; and
a processor that analyzes information related to the acoustical response of the vehicle tire as the vehicle tire rolls over the surface received by the receiver and detects irregularities in the vehicle.

21. The system of claim 20, wherein the system detects the presence of material not part of the originally manufactured vehicle hidden within the vehicle.

22. The system of claim 20, wherein the system detects abnormal tire pressure without physically contacting the tire.

23. The system of claim 20, wherein the system includes a vibrometer.

24. The system of claim 20, wherein the system detects the presence, absence, or shifting of a particular acoustical mode of the tire.

25. The system of claim 20, wherein the system indicates the presence of an irregularity in the vehicle when the difference in the Quality Factor between the structural frequencies of the tire and the structural frequencies of a properly inflated tire differ by more than 10%.

26. The system of claim 20, wherein the irregularity detected by the system is the presence of material not part of the originally manufactured vehicle hidden within the vehicle.

27. A system, comprising:
a camera for capturing video images of a vehicle; and
a processor that analyzes the video images of a vehicle and detects irregularities in the vehicle.

28. The system of claim 27, wherein the system analyzes the orientation of the body of the vehicle.

29. The system of claim 28, wherein the system analyzes whether the vehicle is oriented in a front end up or front end down orientation when compared with the a normally loaded vehicle.

30. The system of claim 27, wherein the system analyzes the shape of at least one of the vehicle's tires.

31. The system of claim 27, wherein the system analyzes the type, make, or model of the vehicle.

32. The system of claim 27, wherein the irregularity detected by the system is the presence of material not part of the originally manufactured vehicle hidden within the vehicle.

33. A system, comprising:
a sensor that receives information about a the kinematic response of a vehicle component to external excitation; and
a processor that analyzes the information and detects irregularities in the vehicle.

34. The system of claim 33, wherein the system detects irregularities in a vehicle by analyzing the kinematic response of the vehicle moving over a surface.

35. The system of claim 34, wherein the system includes a textured surface over which the vehicle moves as the vehicle's kinematic response to the textured surface is analyzed.

36. The system of claim 35, wherein the textured surface includes a bump over which at least one vehicle wheel travels.

37. The system of claim 35, wherein the surface over which the vehicle wheel travels includes the sensor.
